SEQUENCE LISTING (44 sequences)

SEQ ID NO: 1 - protein, 70 aa, Artificial
  Synthetic
XXXXXXXXXXXXXXXXXXXXXXXXXXXXXXXXXXXXXXXXXXXXXXXXXXXXXXXXXXXXXXXXXXXXXX

SEQ ID NO: 2 - protein, 70 aa, Artificial
  Synthetic
WLRRIKAXXXXXXXXXXXXXXXXXXXXXXXXXXXXXXXXXXXXXXXXXXXXXXXXXXXXXXXXXXXXXXX

SEQ ID NO: 3 - protein, 7 aa, Artificial
  Synthetic
WLRRIKA

SEQ ID NO: 4 - protein, 14 aa, Artificial
  Synthetic
WLRRIKAWLRRIKA

SEQ ID NO: 5 - protein, 21 aa, Artificial
  Synthetic
WLRRIKAWLRRIKAWLRRIKA

SEQ ID NO: 6 - protein, 160 aa, Artificial
  Synthetic
MEIPVPVQPSWLRRASAPLPGLSAPGRLFDQRFGEGLLEAELAALCPTTLAPYYLRAPSVALPVAQVPTDPGHFSVLLDVKHFSPEEIAVKVVGEHVEVHARHEERPDEHGFVAREFHRRYRLPPGVDPAAVTSALSPEGVLSIQAAPASAQAPPPAAAK

SEQ ID NO: 7 - protein, 13 aa, Artificial
  Synthetic
XXXXAXAPLPXXX

SEQ ID NO: 8 - protein, 4 aa, Artificial
  Synthetic
WLRR

SEQ ID NO: 9 - protein, 13 aa, Artificial
  Synthetic
WLRRASAPLPGLK

SEQ ID NO: 10 - protein, 20 aa, Artificial
  Synthetic
WLRRIKAWLRRASAPLPGLK

SEQ ID NO: 11 - protein, 27 aa, Artificial
  Synthetic
WLRRIKAWLRRIKAWLRRASAPLPGLK

SEQ ID NO: 12 - protein, 34 aa, Artificial
  Synthetic
WLRRIKAWLRRIKAWLRRIKAWLRRASAPLPGLK

SEQ ID NO: 13 - protein, 8 aa, Artificial
  Synthetic
XXRRAXAP

SEQ ID NO: 14 - protein, 6 aa, Artificial
  Synthetic
RRASAP

SEQ ID NO: 15 - protein, 13 aa, Artificial
  Synthetic
WLRRIKARRASAP

SEQ ID NO: 16 - protein, 20 aa, Artificial
  Synthetic
WLRRIKAWLRRIKARRASAP

SEQ ID NO: 17 - protein, 27 aa, Artificial
  Synthetic
WLRRIKAWLRRIKAWLRRIKARRASAP

SEQ ID NO: 18 - protein, 13 aa, Artificial
  Synthetic
GRKKRRQRRRPPQ

SEQ ID NO: 19 - protein, 11 aa, Artificial
  Synthetic
YARAAARQARA

SEQ ID NO: 20 - protein, 34 aa, Artificial
  Synthetic
DAATATRGRSAASRPTERPRAPARSASRPRRPVE

SEQ ID NO: 21 - protein, 27 aa, Artificial
  Synthetic
GWTLNSAGYLLGLINLKALAALAKKIL

SEQ ID NO: 22 - protein, 12 aa, Artificial
  Synthetic
PLSSIFSRIGDP

SEQ ID NO: 23 - protein, 16 aa, Artificial
  Synthetic
AAVALLPAVLLALLAP

SEQ ID NO: 24 - protein, 12 aa, Artificial
  Synthetic
AAVLLPVLLAAP

SEQ ID NO: 25 - protein, 15 aa, Artificial
  Synthetic
VTVLALGALAGVGVG

SEQ ID NO: 26 - protein, 21 aa, Artificial
  Synthetic
GALFLGWLGAAGSTMGAWSQP

SEQ ID NO: 27 - protein, 27 aa, Artificial
  Synthetic
GWTLNSAGYLLGLINLKALAALAKKIL

SEQ ID NO: 28 - protein, 18 aa, Artificial
  Synthetic
KLALKLALKALKAALKLA

SEQ ID NO: 29 - protein, 21 aa, Artificial
  Synthetic
KETWWETWWTEWSQPKKKRKV

SEQ ID NO: 30 - protein, 16 aa, Artificial
  Synthetic
KAFAKLAARLYRKAGC

SEQ ID NO: 31 - protein, 16 aa, Artificial
  Synthetic
KAFAKLAARLYRAAGC

SEQ ID NO: 32 - protein, 16 aa, Artificial
  Synthetic
AAFAKLAARLYRKAGC

SEQ ID NO: 33 - protein, 16 aa, Artificial
  Synthetic
KAFAALAARLYRKAGC

SEQ ID NO: 34 - protein, 16 aa, Artificial
  Synthetic
KAFAKLAAQLYRKAGC

SEQ ID NO: 35 - protein, 15 aa, Artificial
  Synthetic
GGGGYGRKKRRQRRR

SEQ ID NO: 36 - protein, 11 aa, Artificial
  Synthetic
YGRKKRRQRRR

SEQ ID NO: 37 - protein, 8 aa, Artificial
  Synthetic
XWLRRIKA

SEQ ID NO: 38 - protein, 15 aa, Artificial
  Synthetic
XWLRRIKAWLRRIKA

SEQ ID NO: 39 - protein, 22 aa, Artificial
  Synthetic
XWLRRIKAWLRRIKAWLRRIKA

SEQ ID NO: 40 - protein, 9 aa, Artificial
  Synthetic
RRRRXXXXX

SEQ ID NO: 41 - protein, 12 aa, Artificial
  Synthetic
YKALRISRKLAK

SEQ ID NO: 42 - protein, 24 aa, Artificial
  Synthetic
YARAAARQARAWLRRASAPLPGLK

SEQ ID NO: 43 - protein, 24 aa, Artificial
  Synthetic
YGRKKRRQRRRWLRRASAPLPGLK

SEQ ID NO: 44 - protein, 21 aa, Artificial
  Synthetic
XWLRRIKAWLRRASAPLPGLK

(19) United States
(12) Patent Application Publication
(10) Pub. No.: US 2010/0004165 A1
(43) Pub. Date: Jan. 7, 2010

(54) Novel Protein Transduction Domains and Uses Therefor

(75) Inventors: Colleen Brophy, Nashville, TN (US); Alyssa Panitch, W. Lafayette, IN (US); Elizabeth Furnish, Tempe, AZ (US); Brandon Seal, Mesa, AZ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: The Arizona Board of Regents, a body corporate acting on behalf of Arizona State University, Scottsdale, AZ (US)

(21) Appl. No.: 12/090,474

(22) PCT Filed: Oct. 30, 2006
(86) PCT No.: PCT/US06/42209

§ 371 Date: Feb. 4, 2009

Related U.S. Application Data
(60) Provisional application No. 60/732,365, filed on Nov. 1, 2005.

(51) Int. Cl. A61K 38/16; C07K 7/06; C07K 7/08; C07K 14/00; A61K 38/08; A61K 38/10; C07H 21/00; C12N 15/74; C12N 1/00; A61P 35/00

(52) U.S. Cl. 514/12; 530/329; 530/328; 530/327; 530/326; 530/325; 530/324; 530/350; 514/16; 514/15; 514/14; 536/23.1; 435/320.1; 435/243

(57) ABSTRACT

The present invention provides novel transduction domains, compositions comprising such transduction domains, and their use for in vivo molecular delivery.

### CROSS REFERENCE

This application claims priority from U.S. Provisional Patent Application Ser. No. 60/732,365 filed Nov. 1, 2005, the disclosure of which is incorporated by reference herein in its entirety.

### STATEMENT OF GOVERNMENT INTEREST

The U.S. Government through the National Institute of Health provided financial assistance for this project under Grant No. RO1 HL58027. Therefore, the United States Government may own certain rights to this invention.

### BACKGROUND

Protein transduction domains (PTDs), also known as cell penetrating peptides, are a class of small peptides capable of penetrating the plasma membrane of mammalian cells [6]. There are several well known PTDs: the HIV transcription factor TAT (SEQ ID NO: 43), the Antp peptide derived from the Drosophila melanogaster homeodomain protein, the herpes simplex virus protein VP22, and arginine oligomers [7-9]. These peptides have been reported to transport compounds of many types and molecular weights, such as conjugated peptides, oligonucleotides, and small particles such as liposomes across mammalian cells [9, 11-13]. Thus, PTDs represent an important class of drug delivery devices, and it is desirable in the art to provide further PTDs for use in drug delivery.

### SUMMARY OF THE INVENTION

In a first aspect, the present invention provides polypeptides comprising an amino acid sequence according to general formula 1:

| | |
|---|---|
| (X₁X₂B₁B₂X₃B₃X₄)ₙ | (SEQ ID NO: 1) |

wherein X₁-X₄ are independently any hydrophobic amino acid;

wherein B₁, B₂, and B₃ are independently any basic amino acid; and

wherein n is between 1 and 10.

In various preferred embodiments, B₁ and B₂, and B₃ are independently arginine or lysine. In a further preferred embodiment, n is between 1 and 3.

In a second aspect, the present invention provides compositions, comprising a polypeptide of the invention combined with a cargo comprising a therapeutically active molecule or compound. In various embodiments, the polypeptide and cargo can be covalently bound, or can be unlinked. In a preferred embodiment, the composition comprises an HSP20 composition.

In a third aspect, the present invention provides pharmaceutical compositions, comprising one or more polypeptides of the present invention and a pharmaceutically acceptable carrier.

In a fourth aspect, the present invention provides isolated nucleic acid sequences encoding a polypeptide of the present invention. In fifth and sixth aspects, the present invention provides recombinant expression vectors comprising the nucleic acid sequences of the present invention, and host cells transfected with the recombinant expression vectors of the present invention, respectively.

In a seventh aspect, the invention provides improved biomedical devices, wherein the biomedical devices comprise one or more polypeptides of the present invention disposed on or in the biomedical device. In various embodiments, such biomedical devices include stents, grafts, shunts, stent grafts, angioplasty devices, balloon catheters, fistulas, wound dressings, and any implantable drug delivery device.

In an eighth aspect, the present invention provides methods for drug delivery, comprising preparing a composition according to the present invention and using it to deliver the cargo as appropriate to an individual in need of the treatment using the cargo. In various embodiments of this eighth aspect, the present invention provides methods for one or more of the following therapeutic uses

(a) inhibiting smooth muscle cell proliferation and/or migration; (b) promoting smooth muscle relaxation; (c) increasing the contractile rate in heart muscle; (d) increasing the rate of heart muscle relaxation; (e) promoting wound healing; (f) reducing scar formation; (g) disrupting focal adhesions; (h) regulating actin polymerization; and (i) treating or inhibiting one or more of intimal hyperplasia, stenosis, restenosis, atherosclerosis, smooth muscle cell tumors, smooth muscle spasm, angina, Prinzmetal's angina (coronary vasospasm), ischemia, stroke, bradycardia, hypertension, pulmonary (lung) hypertension, asthma (bronchospasm), toxemia of pregnancy, pre-term labor, pre-eclampsia/eclampsia, Raynaud's disease or phenomenon, hemolytic-uremia, non-occlusive mesenteric ischemia, anal fissure, achalasia, impotence, migraine, ischemic muscle injury associated with smooth muscle spasm, vasculopathy, such as transplant vasculopathy, bradyarrythmia, bradycardia, congestive heart failure, stunned myocardium, pulmonary hypertension, and diastolic dysfunction;

wherein the method comprises administering to a subject in need thereof an effective amount to carry out the one or more therapeutic uses of an HSP20 composition.

In a ninth aspect, the present invention provides methods for topical or transdermal delivery of an active cargo, comprising combining a transduction domain and an active cargo, where the cargo is not covalently bound to the transduction domain, and contacting the skin of a subject to whom the active agent is to be delivered, wherein the active cargo is delivered through the skin of the subject.

### BRIEF DESCRIPTION OF THE FIGURES

FIG. 1. (A) PTD or W³ (non-covalently bound) transduction (B) Skin penetration [E+D] when 1 mM W3 was used to carry P20 (SEQ ID NO: 9)(non-covalently bound). (C) Skin penetration with P20 (SEQ ID NO: 9) was conjugated to PTD or W1 or when W3 was used alone.

FIG. 2. In vitro peptide penetration in the SC, [E+D], and their transdermal delivery after 4 h using PBS or formulations containing the penetration enhancers monoolein (MO, 10% w/w) or oleic acid (OA, 5% w/w). The number of replicates is 4-8 per experimental group. *, p<0.05 compared to propylene glycol solution. PL: propylene glycol, SC: stratum corneum, [E+D]: epidermis without stratum corneum plus dermis.

FIG. 3. Time-course of in vitro peptide penetration in the SC (A-C), [E+D] (D-F) and whole skin (G-I) after 0.5, 1, 2, 4 or 8 h. The figure also shows the rate of skin penetration, calculated using the penetration of the peptides in the whole skin (J-L). The number of replicates is 6-8 per experimental group. SC: stratum corneum, [E+D]: epidermis without stratum corneum plus dermis. When P20 (SEQ ID NO: 9) was conjugated to YARA (PTD) (SEQ ID NO: 19) and TAT (SEQ ID NO: 43), its penetration in both SC and [E+D] was significantly (p<0.05) higher than that of nonconjugated P20 (SEQ ID NO: 9) at all time points studied.

FIG. 4. WL-P20 (SEQ ID NO: 10) relaxes smooth muscle. Rat aorta was pre-contracted with KCl (110 mM) and then treated with 1 mM WL-P20 (SEQ ID NO: 10). Maximum relaxation (88%) occurred at ˜60 minutes.

FIG. 5. CTGF and collagen expression after TGFβ1 treatment.

### DETAILED DESCRIPTION OF THE INVENTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can occur, and that the description includes instances where said event or circumstance occurs and instances where it does not.

Both single letter and three letter amino acid abbreviations are used within the application. As is well known by one of skill in the art, such single letter designations are as follows:

A is alanine; C is cysteine; D is aspartic acid; E is glutamic acid; F is phenylalanine; G is glycine; H is histidine; I is isoleucine; K is lysine; L is leucine; M is methionine; N is asparagine; P is proline; Q is gluatamine; R is arginine; S is serine; T is threonine; V is valine; W is tryptophan; and Y is tyrosine.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

In a first aspect, the present invention provides polypeptides comprising or consisting of an amino acid sequence according to general formula 1:

| | |
|---|---|
| (X₁X₂B₁B₂X₃B₃X₄)ₙ | (SEQ ID NO: 1) |

wherein X₁-X₄ are independently any hydrophobic amino acid;

wherein B₁, B₂, and B₃ are independently any basic amino acid; and

wherein n is between 1 and 10.

Thus, “n” can be 1, 2, 3, 4, 5, 6, 7, 8, 9, or 10. In a preferred embodiment, n is 1, 2, or 3.

These polypeptides have been shown to be useful for preparing the compositions of the invention (see below), and/or for providing transport of the compositions across mammalian cell membranes.

In a further preferred embodiment of this first aspect, X₁-X₄ are independently any hydrophobic amino acid selected from the group consisting of Trp, Tyr, Leu, Ile, Phe, Val, Met, Cys, Pro, and Ala; and

B₁, B₂, and B₃ are independently arginine, histidine, or lysine.

In various preferred embodiments of this first aspect, both B₁ and B₂ are arginine or lysine and B₃ is either lysine or arginine but is not the same as B₁ and B₂. In a most preferred embodiment, B₁ and B₂ are arginine and B₃ is lysine.

In further preferred embodiments of this first aspect, X₁-X₄ are independently selected from the group consisting of Trp, Leu, Ile, and Ala. In various further preferred embodiments of the first aspect of the invention, X₁ is Trp, X₂ is Leu, X₃ is Ile, or X₄ is Ala, or any combination thereof.

Polypeptides according to this general formula are demonstrated herein to be effective as protein transduction domains, and thus to be of use in the delivery of various therapeutic agents across mammalian cell membranes. As is further demonstrated herein, the polypeptides are also capable of transporting therapeutic moieties (“cargo”) across the skin, whether the cargo is covalently linked to the polypeptide or is simply combined with the polypeptide but not physically linked.

The term “polypeptide” is used in its broadest sense to refer to a sequence of subunit amino acids, amino acid analogs, or peptidomimetics. The subunits are linked by peptide bonds, except where noted. The polypeptides described herein may be chemically synthesized or recombinantly expressed. Recombinant expression can be accomplished using standard methods in the art, generally involving the cloning of nucleic acid sequences capable of directing the expression of the polypeptides into an expression vector, which can be used to transfect or transduce a host cell in order to provide the cellular machinery to carry out expression of the polypeptides. Such expression vectors can comprise bacterial or viral expression vectors, and such host cells can be prokaryotic or eukaryotic.

Preferably, the polypeptides for use in the methods of the present invention are chemically synthesized. Synthetic polypeptides, prepared using the well-known techniques of solid phase, liquid phase, or peptide condensation techniques, or any combination thereof, can include natural and unnatural amino acids. Amino acids used for peptide synthesis may, for example, be standard Boc (Nα-amino protected Nα-t-butyloxycarbonyl) amino acid resin with standard deprotecting, neutralization, coupling and wash protocols, or the base-labile Nα-amino protected 9-fluorenylmethoxycarbonyl (Fmoc) amino acids. Both Fmoc and Boc Nα-amino protected amino acids can be obtained from Sigma, Cambridge Research Biochemical, or other chemical companies familiar to those skilled in the art. In addition, the polypeptides can be synthesized with other Nα-protecting groups that are familiar to those skilled in this art.

Solid phase peptide synthesis may be accomplished by techniques familiar to those in the art and provided, or using automated synthesizers. The polypeptides of the invention may comprise D-amino acids (which are resistant to L-amino acid-specific proteases in vivo), a combination of D- and L-amino acids, and various “designer” amino acids (e.g., β-methyl amino acids, Cα-methyl amino acids, and Nα-methyl amino acids, etc.) to convey special properties. Synthetic amino acid analogues include ornithine for lysine, and norleucine for leucine or isoleucine.

In addition, the polypeptides can have peptidomimetic bonds, such as ester bonds, to prepare polypeptides with novel properties. For example, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo.

The polypeptides of the invention may comprise additional amino acid residues at either or both of the amino and carboxy termini, and may further include additional groups, such as detectable labels including but not limited to fluorescein, fluorescein isothiocyanate, fluorescein isothiocyanate-β-alanine, dansyl glycine, dansyl bound to an amino acid, fluorescent labels attached to an acetyl group; protecting groups including but not limited to Fmoc or other N-terminal protecting group (e.g. Boc); and residues for derivatizing the polypeptide, including but not limited to cysteine for specific thiol coupling. In a further embodiment, the polypeptide or a portion thereof may be cyclic.

In a most preferred embodiment, the polypeptides of the first aspect of the invention comprise or consist of the amino acid sequence (WLRRIKA)ₙ (SEQ ID NO: 2), wherein n is 1-10. Thus, in this embodiment the polypeptide can comprise or consist of 1, 2, 3, 4, 5, 6, 7, 8, 9, or 10 copies of WLRRIKA (SEQ ID NO: 3). In preferred embodiments of this most preferred embodiment, n is 1, 2, or 3. Non-limiting examples of such polypeptides include:

| | |
|---|---|
| WLRRIKA; | (SEQ ID NO: 3) |
| WLRRIKAWLRRIKA; | (SEQ ID NO: 4) |
| and | |
| WLRRIKAWLRRIKAWLRRIKA. | (SEQ ID NO: 5) |

The polypeptide genus (X₁X₂B₁B₂X₃B₃X₄)ₙ (SEQ ID NO: 1) was developed around the non-limiting example WLRRIKA (SEQ ID NO: 3). It is hypothesized that basic amino acids or repeats of basic amino acids need to be surrounded by one or more hydrophobic amino acids to provide for or enhance the transduction of a peptide within the described genus. In the example of WLRRIKA (SEQ ID NO: 3), B₁, B₂, and B₃ are arginine, arginine, and lysine, respectively. It is hypothesized that transduction would still occur when positions B1, B2, and B3 are filled with any amino acid with a net positive charge at physiologically relevant pH, such as lysine, arginine, and histidine. Thus, the polypeptide genus was developed to allow for positions B₁, B₂, and B₃ to be filled by the same or different basic amino acid. In the example of WLRRIKA (SEQ ID NO: 3), X₁, X₂, X₃ and X₄ are tryptophan, leucine, isoleucine, and alanine, respectively. Each of these amino acids is hydrophobic, and it is hypothesized that tryptophan, leucine, isoleucine, and alanine could be used in any or all of the positions designated X₁, X₂, X₃ and X₄. It is further hypothesized that any hydrophobic amino acids could be used in positions X₁, X₂, X₃, and X₄ because of the hypothesis that the combination of hydrophobic and basic amino acids promotes or enhances transduction.

In a second aspect, the present invention provides compositions, comprising a polypeptide of the invention and a cargo. As used herein, “cargo” or “cargoes” mean any molecule or compound, including but not limited to peptides of any length, polynucleotides, organic molecules, antibodies, and liposomes. In a preferred embodiment, the cargo is selected from the group consisting of peptide, polynucleotides, and organic molecules. As disclosed herein, the polypeptides of the invention can be used to carry a cargo across mammalian cell membranes, as well as skin. Such activity is shown whether cargo is covalently bound, or is simply combined with a polypeptide of the invention without direct linkage. Such compositions are thus useful, for example, as therapeutics.

In a preferred embodiment of this second aspect, the cargo is covalently bound to the polypeptide. Exemplary cargoes include, but are not limited to radionuclides, fluorescent markers (including but not limited to green fluorescent protein and similar fluorescent proteins), dyes, imaging agents, RNA, DNA, cDNA; aptamers, antisense oligonucleotides, siRNAs, viral nucleic acid sequences, viral polypeptides, vaccines, and other therapeutic cargo, including but not limited to antipyretics, analgesics and antiphlogistics (e.g., indoinethacin, aspirin, diclofenac sodium, ketoprofen, ibuprofen, mefenamic acid, azulene, phenacetin, isopropyl antipyrine, acetaminophen, benzadac, phenylbutazone, flufenamic acid, sodium salicylate, salicylamide, sazapyrine and etodolac); steroidal anti-inflammatory drugs (e.g., dexamethasone, hydrocortisone, prednisolone and triamcinolone); antiulcer drugs (e.g., ecabet sodium, enprostil, sulpiride, cetraxate hydrochloride, gefarnate, irsogladine maleate, cimetidine, ranitidine hydrochloride, famotidine, nizatidine and roxatidine acetate hydrochloride); coronary vasodilators (e.g., nifedipine, isosorbide dinitrate, diltiazem hydrochloride, trapidil, dipyridamole, dilazep hydrochloride, verapamil, nicardipine, nicardipine hydrochloride and verapamil hydrochloride); peripheral vasodilators (e.g., ifenprodil tartrate, cinepacide maleate, ciclandelate, cynnaridine and pentoxyfylline); antibiotics (e.g., ampicillin, amoxicillin, cefalexin, erythromycin ethyl succinate, bacampicillin hydrochloride, minocycline hydrochloride, chloramphenicol, tetracycline, erythromycin, ceftazidime, cefuroxime sodium, aspoxicillin and ritipenem acoxyl hydrate); synthetic antimicrobials (e.g., nalidixic acid, piromidic acid, pipemidic acid trihydrate, enoxacin, cinoxacin, ofloxacin, norfloxacin, ciprofloxacin hydrochloride and sulfamethoxazole-trimethoprim); antiviral agents (e.g., aciclovir and ganciclovir); anticonvulsants (e.g., propantheline bromide, atropine sulfate, oxitropium bromide, timepidium bromide, scopolamine butylbromide, trospium chloride, butropium bromide, N-methylscopolamine methylsulfate and methyloctatropine bromide); antitussives (e.g., tipepidine hibenzate, methylephedrine hydrochloride, codeine phosphate, tranilast, dextromethorphan hydrobromide, dimemorfan phosphate, clofenadol hydrochloride, fominoben hydrochloride, benproperine phosphate, eprazinone hydrochloride, clofedanol hydrochloride, ephedrine hydrochloride, noscapine, pentoxyverine citrate, oxeladin citrate and isoaminyl citrate); expectorants (e.g., bromhexine hydrochloride, carbocysteine, ethyl cysteine hydrochloride and methylcysteine hydrochloride); bronchodilators (e.g., theophylline, aminophylline, sodium cromoglicate, procaterol hydrochloride, trimetoquinol hydrochloride, diprophylline, salbutamol sulfate, clorprenaline hydrochloride, formoterol fumarate, orciprenaline sulfate, pirbuterol hydrochloride, hexoprenaline sulfate, bitolterol mesilate, clenbuterol hydrochloride, terbutaline sulfate, mabuterol hydrochloride, fenoterol hydrobromide and methoxyphenamine hydrochloride); cardiacs (e.g., dopamine hydrochloride, dobutamine hydrochloride, docarpamine, denopamine, caffeine, digoxin, digitoxin and ubidecarenone); diuretics (e.g., furosemide, acetazolamide, trichlormethiazide, methylclothiazide, hydrochlorothiazide, hydroflumethiazide, ethiazide, cyclopenthiazide, spironolactone, triamterene, florothiazide, piretanide, mefruside, etacrynic acid, azosemide and clofenamide); muscle relaxants (e.g., chlorphenesin carbamate, tolperisone hydrochloride, eperisone hydrochloride, tizanidine hydrochloride, mephenesine, chlorzoxazone, phenprobamate, methocarbamol, chlormezanone, pridinol mesilate, afloqualone, baclofen and dantrolene sodium); cerebral metabolism ameliorants (e.g., nicergoline, meclofenoxate hydrochloride and taltireline); minor tranquilizers (e.g., oxazolam, diazepam, clotiazepam, medazepam, temazepaam, fludiazepam, meprobamate, nitrazepam and chlordiazepoxide); major tranquilizers (e.g., sulpiride, clocapramine hydrochloride, zotepine, chlorpromazine and haloperidol); beta-blockers (e.g., bisoprolol fumarate, pindolol, propranolol hydrochloride, carteolol hydrochloride, metoprolol tartrate, labetanol hydrochloride, acebutolol hydrochloride, bufetolol hydrochloride, alprenolol hydrochloride, arotinolol hydrochloride, oxprenolol hydrochloride, nadolol, bucumolol hydrochloride, indenolol hydrochloride, timolol maleate, befunolol hydrochloride and bupranolol hydrochloride); antiarrthymics (e.g., procainamide hydrochloride, disopyramide phosphate, cibenzoline succinate, ajmaline, quinidine sulfate, aprindine hydrochloride, propafenone hydrochloride, mexiletine hydrochloride and ajmilide hydrochloride); athrifuges (e.g., allopurinol, probenicid, colistin, sulfinpyrazone, benzbromarone and bucolome); anticoagulants (e.g., ticlopidine hydrochloride, dicumarol, potassium warfarin, and (2R,3R)-3-acetoxy-5-[2(dimethylamino)ethyl]-2,3-dihydro-8-methyl-2-(4-ethylphenyl)-1,5-benzothiazepine-4(5H)-o-ne maleate); thrombolytics (e.g., methyl(2E,3Z)-3-benzylidene-4-(3,5-dimethoxy-.alpha.-methyl benzylidene)-N-(4-methylpiperazin-1-yl)succinamate hydrochloride); liver disease drugs (e.g., (+)-r-5-hydroxymethyl-t-7-(3,4-dimethoxyphenyl)-4-oxo-4,5,6,7-tetrahydro benzo[b]furan-c-6-carboxylactone); antiepileptics (e.g., phenytoin, sodium valproate, metalbital and carbamazepine); antihistamines (e.g., chlorpheniramine maleate, clemastine fumarate, mequitazine, alimemazine tartrate, cyproheptadine hydrochloride and bepotastin besilate); antiemetics (e.g., difenidol hydrochloride, metoclopramide, domperidone and betahistine mesilate and trimebutine maleate); depressors (e.g., dimethylaminoethyl reserpilinate dihydrochloride, rescinnamine, methyldopa, prazocin hydrochloride, bunazosin hydrochloride, clonidine hydrochloride, budralazine, urapidil and N-[6-[2-[(5-bromo-2-pyrimidinyl)oxy]ethoxy]-5-(4-methylphenyl)-4-pyrimidi-nyl]-4-(2-hydroxy-1,1-dimethylethyl)benzene sulfonamide sodium); hyperlipidemia agents (e.g., pravastatin sodium and fluvastatin sodium); sympathetic nervous stimulants (e.g., dihydroergotamine mesilate and isoproterenol hydrochloride, etilefrine hydrochloride); oral diabetes therapeutic drugs (e.g., glibenclamide, tolbutamide and glymidine sodium); oral carcinostatics (e.g., marimastat); vitamins (e.g., vitamin B1, vitamin B2, vitamin B6, vitamin B12, vitamin C and folic acid); thamuria therapeutic drugs (e.g., flavoxate hydrochloride, oxybutynin hydrochloride and terolidine hydrochloride); angiotensin convertase inhibitors (e.g., imidapril hydrochloride, enalapril maleate, alacepril and delapril hydrochloride), HSP20, TGF-β, cofilin, 14-3-3, PKA kinase inhibitors, leptin, INF-α, cyclosporin, bacitracin, and palmytoyl-glycyl-hystidyl-lysine tripeptide.

In a further embodiment of this second aspect of the invention, the cargo comprises a peptide therapeutic. In one particularly preferred embodiment, the cargo is HSP20, a peptide derived therefrom, or an analogue thereof (collectively referred to as “HSP20 peptide”), and the composition is referred to as an “HSP20 composition.” In one embodiment, the HSP peptide portion of the HSP20 composition comprises or consists of full length HSP20:

(SEQ ID NO: 6)

Met Glu Ile Pro Val Pro Val Gln Pro Ser Trp Leu
Arg Arg Ala Ser Ala Pro Leu Pro Gly Leu Ser Ala
Pro Gly Arg Leu Phe Asp Gln Arg Phe Gly Glu Gly
Leu Leu Glu Ala Glu Leu Ala Ala Leu Cys Pro Thr
Thr Leu Ala Pro Tyr Tyr Leu Arg Ala Pro Ser Val
Ala Leu Pro Val Ala Gln Val Pro Thr Asp Pro Gly
His Phe Ser Val Leu Leu Asp Val Lys His Phe Ser
Pro Glu Glu Ile Ala Val Lys Val Val Gly Glu His
Val Glu Val His Ala Arg His Glu Glu Arg Pro Asp
Glu His Gly Phe Val Ala Arg Glu Phe His Arg Arg
Tyr Arg Leu Pro Pro Gly Val Asp Pro Ala Ala Val
Thr Ser Ala Leu Ser Pro Glu Gly Val Leu Ser Ile
Gln Ala Ala Pro Ala Ser Ala GIn Ala Pro Pro Pro
Ala Ala Ala Lys.

In another embodiment the HSP20 peptide portion of the HSP20 composition comprises or consists of an amino acid sequence of formula 1:

| | |
|---|---|
| X3-A(X4)APLP-X5 | (SEQ ID NO: 7) |

wherein X3 is 0, 1, 2, 3, or 4 amino acids of the sequence WLRR (SEQ ID NO: 8);

X4 is selected from the group consisting of S, T, Y, D, E, hydroxylysine, hydroxyproline, phosphoserine analogs and phosphotyrosine analogs;

X5 is 0, 1, 2, or 3 amino acids of a sequence of genus Z1-Z2-Z3,
wherein Z1 is selected from the group consisting of G and D;

Z2 is selected from the group consisting of L and K; and

Z3 is selected from the group consisting of K, S and T.

It is more preferred in this embodiment that X4 is S, T, or Y; more preferred that X4 is S or T, and most preferred that X4 is S. In these embodiments where X4 is S, T, or Y, it is most preferred that X4 is phosphorylated. When X4 is D or E, these residues have a negative charge that mimics the phosphorylated state. HSP20 peptides are optimally effective in the methods of the invention when X4 is phosphorylated, is a phosphoserine or phosphotyrosine mimic, or is another mimic of a phosphorylated amino acid residue, such as a D or E residue. Examples of phosphoserine mimics include, but are not limited to, sulfoserine, amino acid mimics containing a methylene substitution for the phosphate oxygen, 4-phosphono(difluoromethyl)phenylanaline, and L-2-amino-4-(phosphono)-4,4-difuorobutanoic acid. Other phosphoserine mimics can be made by those of skill in the art; for example, see Otaka et al., Tetrahedron Letters 36:927-930 (1995). Examples of phosphotyrosine mimics include, but are not limited to, phosphonomethylphenylalanine, difluorophosphonomethylphenylalanine, fluoro-O-malonyltyrosine and O-malonyltyrosine. (See, for example, Akamatsu et. al., Bioorg Med Chem January 1997;5(1):157-63).

In a most preferred embodiment of formula 1, the HSP20 peptide comprises or consists of WLRRAS*APLPGLK (SEQ ID NO: 9), wherein S* represents a phosphorylated serine residue. In this embodiment, the HSP20 composition preferably comprises or consists of an amino acid sequence selected from:

(SEQ ID NO: 10)

WLRRIKAWLRRAS*APLPGLK;

(SEQ ID NO: 11)

WLRRIKAWLRRIKAWLRRAS*APLPGLK;
and

(SEQ ID NO: 12)

WLRRIKAWLRRIKAWLRRIKAWLRRAS*APLPGLK.

In another embodiment of the HSP20 compositions, the HSP20 peptide comprises or consists of an amino acid sequence of formula 2:

| | |
|---|---|
| X2-X3-RRA-X4-AP | (SEQ ID NO: 13) |

Wherein X2 is absent or is W;

X3 is absent or is L; and

X4 is selected from the group consisting of S, T, Y, D, E, phosphoserine analogs and phosphotyrosine analogs (with preferred embodiments as described for formula 1).

In a most preferred embodiment of formula 2, the HSP20 peptide comprises or consists of RRAS*AP (SEQ ID NO: 14), wherein S* represents a phosphorylated serine residue. In this embodiment, the HSP20 composition preferably comprises or consists of an amino acid sequence selected from:

| | |
|---|---|
| WLRRIKARRAS*AP; | (SEQ ID NO: 15) |
| WLRRIKAWLRRIKARRAS*AP; | (SEQ ID NO: 16) |
| and | |
| WLRRIKAWLRRIKAWLRRIKARRAS*AP. | (SEQ ID NO: 17) |

The polypeptides and/or compositions may be subjected to conventional pharmaceutical operations such as sterilization and/or may contain conventional adjuvants, such as preservatives, stabilizers, wetting agents, emulsifiers, buffers etc

In third aspect, the present invention provides pharmaceutical compositions comprising a polypeptide of the invention and a pharmaceutically acceptable carrier, or a composition of the invention and a pharmaceutically acceptable carrier. Such pharmaceutical compositions are especially useful for carrying out the methods of the invention described below.

For administration, the polypeptides or compositions are ordinarily combined with one or more adjuvants appropriate for the indicated route of administration. The polypeptides or compositions may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, dextran sulfate, heparin-containing gels, and/or polyvinyl alcohol, and tableted or encapsulated for conventional administration. Alternatively, the polypeptides or compositions may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art. The polypeptides or compositions may be linked to other compounds to promote an increased half-life in vivo, such as polyethylene glycol. Such linkage can be covalent or non-covalent as is understood by those of skill in the art.

The pharmaceutical compositions may be administered by any suitable route, including oral, parental, by inhalation spray, transdermal, transmucosal, rectal, vaginal, or topical routes in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Preferred embodiments for administration vary with respect to the condition being treated.

The pharmaceutical compositions may be made up in a solid form (including granules, powders or suppositories), ointment, or in a liquid form (e.g., solutions, suspensions, or emulsions). The pharmaceutical compositions may be applied in a variety of solutions. Suitable solutions for use in accordance with the invention are sterile, dissolve sufficient amounts of the polypeptides or compositions, and are not harmful for the proposed application.

In fourth aspect, the present invention provides isolated nucleic acids encoding polypeptides or compositions of the present invention. Appropriate nucleic acids according to this aspect of the invention will be apparent to one of skill in the art based on the disclosure provided herein and the general level of skill in the art.

In fifth aspect, the present invention provides expression vectors comprising DNA control sequences operably linked to the isolated nucleic acids of the fourth aspect of the present invention. “Control sequences” operably linked to the nucleic acids of the invention are those nucleic acids capable of effecting the expression of the nucleic acids of the invention. The control sequences need not be contiguous with the nucleic acids, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors.

In a sixth aspect, the present invention provides genetically engineered host cells comprising the expression vectors of the invention. Such host cells can be prokaryotic cells or eukaryotic cells, and can be either transiently or stably transfected, or can be transduced with viral vectors. Such host cells can be used, for example, to produce large amounts of the polypeptides or compositions of the invention.

In a seventh aspect, the invention provides improved biomedical devices, wherein the biomedical devices comprise polypeptides or compositions of the present invention disposed on or in the biomedical device. In a preferred embodiment, the biomedical device comprises an HSP20 composition as disclosed above. As used herein, a “biomedical device” refers to a device to be implanted into or contacted with a subject, for example, a human being, in order to bring about a desired result. Particularly preferred biomedical devices according to this aspect of the invention include, but are not limited to, stents, grafts, shunts, stent grafts, fistulas, angioplasty devices, balloon catheters, implantable drug delivery devices, wound dressings such as films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), cellophane, and biological polymers.

As used herein, the term “grafts” refers to both natural and prosthetic grafts and implants. In a most preferred embodiment, the graft is a vascular graft.

As used herein, the term “stent” includes the stent itself, as well as any sleeve or other component that may be used to facilitate stent placement.

As used herein, “disposed on or in” means that the polypeptides or compositions can be either directly or indirectly in contact with an outer surface, an inner surface, or embedded within the biomedical device. “Direct” contact refers to disposition of the polypeptides or compositions directly on or in the device, including but not limited to soaking a biomedical device in a solution containing the polypeptide or composition, spin coating or spraying a solution containing the polypeptide or composition onto the device, implanting any device that would deliver the polypeptide or composition, and administering the polypeptide or composition through a catheter directly on to the surface or into any organ.

“Indirect” contact means that the polypeptide or composition does not directly contact the biomedical device. For example, the polypeptide or composition may be disposed in a matrix, such as a gel matrix or a viscous fluid, which is disposed on the biomedical device. Such matrices can be prepared to, for example, modify the binding and release properties of the polypeptide or composition as required.

In an eighth aspect, the present invention provides methods for drug delivery, comprising preparing a composition according to the present invention and using it to deliver the cargo as appropriate to an individual in need of the treatment using the cargo. Such “cargo” or “cargoes” can be any compound or molecule, as described in the second aspect of the invention.

In a specific embodiment, the cargo comprises an HSP20 peptide, and the method thus comprises treating the individual with an HSP20 composition as disclosed herein. The inventors have previously demonstrated that HSP20 and peptides derived therefrom show promise as therapeutic agents for the following: (a) inhibiting smooth muscle cell proliferation and/or migration; (b) promoting smooth muscle relaxation; (c) increasing the contractile rate in heart muscle; (d) increasing the rate of heart muscle relaxation; (e) promoting wound healing; (f) reducing scar formation; (g) disrupting focal adhesions; (h) regulating actin polymerization; and (i) treating or inhibiting one or more of intimal hyperplasia, stenosis, restenosis, atherosclerosis, smooth muscle cell tumors, smooth muscle spasm, angina, Prinzmetal's angina (coronary vasospasm), ischemia, stroke, bradycardia, hypertension, pulmonary (lung) hypertension, asthma (bronchospasm), toxemia of pregnancy, pre-term labor, pre-eclampsia/eclampsia, Raynaud's disease or phenomenon, hemolytic-uremia, non-occlusive mesenteric ischemia, anal fissure, achalasia, impotence, female sexual arousal disorder (FSAD), migraine, ischemic muscle injury associated with smooth muscle spasm, vasculopathy, such as transplant vasculopathy, bradyarrythmia, bradycardia, congestive heart failure, stunned myocardium, pulmonary hypertension, and diastolic dysfunction. (See, for example, US 20030060399 filed Mar. 27, 2003; WO2004017912 published Mar. 4, 2004; WO04/075914; WO03/018758; WO05/037236).

Thus, in further embodiments of this aspect, the invention provides methods for one or more of the following therapeutic uses:

(a) inhibiting smooth muscle cell proliferation and/or migration; (b) promoting smooth muscle relaxation; (c) increasing the contractile rate in heart muscle; (d) increasing the rate of heart muscle relaxation; (e) promoting wound healing; (f) reducing scar formation; (g) disrupting focal adhesions; (h) regulating actin polymerization; and (i) treating or inhibiting one or more of intimal hyperplasia, stenosis, restenosis, atherosclerosis, smooth muscle cell tumors, smooth muscle spasm, angina, Prinzmetal's angina (coronary vasospasm), ischemia, stroke, bradycardia, hypertension, pulmonary (lung) hypertension, asthma (bronchospasm), toxemia of pregnancy, pre-term labor, pre-eclampsia/eclampsia, Raynaud's disease or phenomenon, hemolytic-uremia, non-occlusive mesenteric ischemia, anal fissure, achalasia, impotence, female sexual arousal disorder (FSAD), migraine, ischemic muscle injury associated with smooth muscle spasm, vasculopathy, such as transplant vasculopathy, bradyarrythmia, bradycardia, congestive heart failure, stunned myocardium, pulmonary hypertension, and diastolic dysfunction;

wherein the method comprises administering to an individual in need thereof an effective amount to carry out the one or more therapeutic uses of an HSP20 composition according to the present invention. In preferred embodiments, the methods comprise administering to the individual an HSP20 composition according to one of the preferred embodiments disclosed in the second aspect of the invention.

In a preferred embodiment, the individual is a mammal; in a more preferred embodiment, the individual is a human. In a preferred embodiment of all of the methods of the present invention, the HSP20 peptide is phosphorylated, as disclosed above.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, the term “inhibit” or “inhibiting” means to limit the disorder in individuals at risk of developing the disorder.

As used herein, “administering” includes in vivo administration, as well as administration directly to tissue ex vivo, such as vein grafts.

Intimal hyperplasia is a complex process that leads to graft failure, and is the most common cause of failure of arterial bypass grafts. While incompletely understood, intimal hyperplasia is mediated by a sequence of events that include endothelial cell injury and subsequent vascular smooth muscle proliferation and migration from the media to the intima. This process is associated with a phenotypic modulation of the smooth muscle cells from a contractile to a synthetic phenotype. The “synthetic” smooth muscle cells secrete extracellular matrix proteins, which leads to pathologic narrowing of the vessel lumen leading to graft stenoses and ultimately graft failure. Such endothelial cell injury and subsequent smooth muscle cell proliferation and migration into the intima also characterizes restenosis, most commonly after angioplasty to clear an obstructed blood vessel.

In some embodiments of the methods of the invention, such as those relating to inhibiting smooth muscle cell proliferation and/or migration, or promoting smooth muscle relaxation, the administering may be direct, by contacting a blood vessel in a subject being treated with one or more polypeptides of the invention. For example, a liquid preparation of an HSP20 composition can be forced through a porous catheter, or otherwise injected through a catheter to the injured site, or a gel or viscous liquid containing the HSP20 composition can be spread on the injured site. In these embodiment of direct delivery, it is most preferred that the HSP20 composition be delivered into smooth muscle cells at the site of injury or intervention. This can be accomplished, for example, by delivering the recombinant expression vectors (most preferably a viral vector, such as an adenoviral vector) of the invention to the site, or by directly delivering the HSP20 composition to the smooth muscle cells.

In various other preferred embodiments of this methods of the invention, particularly those that involve inhibiting smooth muscle cell proliferation and/or migration, the method is performed on a subject who has undergone, is undergoing, or will undergo a procedure selected from the group consisting of angioplasty, vascular stent placement, endarterectomy, atherectomy, bypass surgery (such as coronary artery bypass surgery; peripheral vascular bypass surgeries), vascular grafting, organ transplant, prosthetic device implanting, microvascular reconstructions, plastic surgical flap construction, and catheter emplacement.

HSP20, and polypeptides derived therefrom, have been shown to disrupt actin stress fiber formation and adhesion plaques, each of which have been implicated in intimal hyperplasia (see US 20030060399). The data further demonstrate a direct inhibitory effect of the HSP20 polypeptides on intimal hyperplasia (see US 20030060399). Thus, in another embodiment, the methods comprise treating or inhibiting one or more disorder selected from the group consisting of intimal hyperplasia, stenosis, restenosis, and atherosclerosis, comprising contacting a subject in need thereof with an amount effective to treat or inhibit intimal hyperplasia, stenosis, restenosis, and/or atherosclerosis of an HSP20 composition according to the invention.

In a further embodiment of this aspect of the invention, the method is used to treat smooth muscle cell tumors. In a preferred embodiment, the tumor is a leiomyosarcoma, which is defined as a malignant neoplasm that arises from muscle. Since leiomyosarcomas can arise from the walls of both small and large blood vessels, they can occur anywhere in the body, but peritoneal, uterine, and gastro-intestinal (particularly esophageal) Ieiomyosarcomas are more common. Alternatively, the smooth muscle tumor can be a Ieiomyoma, a non-malignant smooth muscle neoplasm. In a further embodiment, the method can be combined with other treatments for smooth muscle cell tumors, such as chemotherapy, radiation therapy, and surgery to remove the tumor.

In a further embodiment, the methods of the invention are used for treating or inhibiting smooth muscle spasm, comprising contacting a subject or graft in need thereof with an amount effective to inhibit smooth muscle spasm of an HSP20 composition according to the invention.

It has been shown that HSP20, and peptides derived therefrom, are effective at inhibiting smooth muscle spasm, such as vasospasm, and may exert their anti-smooth muscle spasm effect by promoting smooth muscle vasorelaxation and inhibiting contraction (see US 20030060399 filed Mar. 27, 2003).

Smooth muscles are found in the walls of blood vessels, airways, the gastrointestinal tract, and the genitourinary tract. Pathologic tonic contraction of smooth muscle constitutes spasm. Many pathological conditions are associated with spasm of vascular smooth muscle (“vasospasm”), the smooth muscle that lines blood vessels. This can cause symptoms such as angina and ischemia (if a heart artery is involved), or stroke as in the case of subarachnoid hemorrhage induced vasospasm if a brain vessel is involved. Hypertension (high blood pressure) is caused by excessive vasoconstriction, as well as thickening, of the vessel wall, particularly in the smaller vessels of the circulation.

Thus, in a further embodiment of the methods of the invention, the muscle cell spasm comprises a vasospasm, and the method is used to treat or inhibit vasospasm. Preferred embodiments of the method include, but are not limited to, methods to treat or inhibit angina, coronary vasospasm, Prinzmetal's angina (episodic focal spasm of an epicardial coronary artery), ischemia, stroke, bradycardia, and hypertension.

In another embodiment of the methods of the invention, smooth muscle spasm is inhibited by treatment of a graft, such as a vein or arterial graft, with an HSP20 composition according to the invention. One of the ideal conduits for peripheral vascular and coronary reconstruction is the greater saphenous vein. However, the surgical manipulation during harvest of the conduit often leads to vasospasm. The exact etiology of vasospasm is complex and most likely multifactorial. Most investigations have suggested that vasospasm is either due to enhanced constriction or impaired relaxation of the vascular smooth muscle in the media of the vein. Numerous vasoconstricting agents such as endothelin-1 and thromboxane are increased during surgery and result in vascular smooth muscle contraction. Other vasoconstrictors such as norepinephrine, 5-hydroxytryptamine, acetylcholine, histamine, angiotensin II, and phenylephrine have been implicated in vein graft spasm. Papaverine is a smooth muscle vasodilator that has been used. In circumstances where spasm occurs even in the presence of papaverine, surgeons use intraluminal mechanical distension to break the spasm. This leads to injury to the vein graft wall and subsequent intimal hyperplasia. Intimal hyperplasia is the leading cause of graft failure.

Thus, in this embodiment, the graft can be contacted with an HSP20 composition according to the invention, during harvest from the graft donor, subsequent to harvest (before implantation), and/or during implantation into the graft recipient (ie: ex vitro or in vivo). This can be accomplished, for example, by delivering the recombinant expression vectors (most preferably a viral vector, such as an adenoviral vector) of the invention to the site, and transfecting the smooth muscle cells, or by direct delivery of the HSP20 composition into smooth muscle. During graft implantation, it is preferred that the subject be treated systemically with heparin, as heparin has been shown to bind to protein transduction domains and prevent them from transducing into cells. This approach will lead to localized protein transduction of the graft alone, and not into peripheral tissues. The methods of this embodiment of the invention inhibit vein graft spasm during harvest and/or implantation of the graft, and thus improve both short and long term graft success.

In various other embodiments of the methods of the invention, the muscle cell spasm is associated with a disorder including, but not limited to pulmonary (lung) hypertension, asthma (bronchospasm), toxemia of pregnancy, pre-term labor, pre-eclampsia/eclampsia, Raynaud's disease or phenomenon, hemolytic-uremia, non-occlusive mesenteric ischemia (ischemia of the intestines that is caused by inadequate blood flow to the intestines), anal fissure (which is caused by persistent spasm of the internal anal sphincter), achalasia (which is caused by persistent spasm of the lower esophageal sphincter), impotence (which is caused by a lack of relaxation of the vessels in the penis; erection requires vasodilation of the corpra cavernosal (penile) blood vessels), migraine (which is caused by spasm of the intracranial blood vessels), ischemic muscle injury associated with smooth muscle spasm, and vasculopathy, such as transplant vasculopathy (a reaction in the transplanted vessels which is similar to atherosclerosis, it involves constrictive remodeling and ultimately obliteration of the transplanted blood vessels: this is the leading cause of heart transplant failure).

Preferred routes of delivery for these various indications of the different embodiments of the methods of the invention vary. Topical administration is preferred for methods involving treatment or inhibition of vein graft spasm, intimal hyperplasia, restenosis, prosthetic graft failure due to intimal hyperplasia, stent, stent graft failure due to intimal hyperplasia/constrictive remodeling, microvascular graft failure due to vasospasm, transplant vasculopathy, and male and female sexual dysfunction. As used herein, “topical administration” refers to delivering the polypeptide or composition onto the surface of the organ.

Intrathecal administration, defined as delivering the polypeptide or composition into the cerebrospinal fluid is the preferred route of delivery for treating or inhibiting stroke and subarachnoid hemorrhage induced vasospasm. Intraperitoneal administration, defined as delivering the polypeptide or composition into the peritoneal cavity, is the preferred route of delivery for treating or inhibiting non-occlusive mesenteric ischemia. Oral administration is the preferred route of delivery for treating or inhibiting achalasia. Intravenous administration is the preferred route of delivery for treating or inhibiting hypertension and bradycardia. Administration via suppository is preferred for treating or inhibiting anal fissure. Aerosol delivery is preferred for treating or inhibiting asthma (ie: bronchospasm). Intrauterine administration is preferred for treating or inhibiting pre-term labor and pre-eclampsia/eclampsia.

In another embodiment of the methods of the invention, the methods are used to increase the contractile rate in heart muscle. Individuals that can benefit from such treatment include those who exhibit a reduced heart rate relative to either a normal heart rate for the individual, or relative to a “normal” heart rate for a similarly situated individual. As used herein, the phrase “increasing the contractile rate in heart muscle” means any increase in contractile rate that provides a therapeutic benefit to the patient. Such a therapeutic benefit can be achieved, for example, by increasing the contractile rate to make it closer to a normal contractile rate for the individual, a normal contractile rate for a similarly situated individual, or some other desired target contractile rate. In a preferred embodiment, the methods result in an increase of at least 5% in the contractile rate of the patient in need of such treatment. In further preferred embodiments, the methods of the invention result in an increase of at least 10%, 15%, 20%, 25%, 30%, 35%, 40%, 45%, and/or 50% in the contractile rate of the patient in need of such treatment. In a preferred embodiment, increasing the contractile rate in heart muscle is accomplished by increasing the heart muscle relaxation rate (ie: if the muscles relax faster they beat faster). In a more preferred embodiment, the methods of the invention result in an increase of at least 5% in the heart muscle relaxation rate of the patient in need of such treatment. In further preferred embodiments, the methods of the invention result in an increase of at least 10%, 15%, 20%, 25%, 30%, 35%, 40%, 45%, and/or 50% in the heart muscle relaxation rate of the patient in need of such treatment.

In a further embodiment of the methods of the invention, the methods are performed to treat one or more cardiac disorders that can benefit from increasing the contractile rate in heart muscle. Such cardiac disorders include bradyarrhythmias, bradycardias, congestive heart failure, pulmonary hypertension, stunned myocardium, and diastolic dysfunction. As used herein, “bradyarrythmia” means an abnormal decrease of the rate of the heartbeat to less than 60 beats per minute, generally cased by a disturbance in the electrical impulses to the heart. A common cause of bradyarrythmias is coronary heart disease, which leads to the formation of atheromas that limit the flow of blood to the cardiac tissue, and thus the cardiac tissue becomes damaged. Bradyarrythmias due to coronary artery disease occur more frequently after myocardial infarction. Symptoms include, but are not limited to, loss of energy, weakness, syncope, and hypotension. As used herein, “Congestive heart failure” means an inability of the heart to pump adequate supplies of blood throughout the body. Such heart failure can be due to a variety of conditions or disorders, including but not limited to hypertension, anemia, hyperthyroidism, heart valve defects including but not limited to aortic stenosis, aortic insufficiency, and tricuspid insufficiency; congenital heart defects including but not limited to coarctation of the aorta, septal defects, pulmonary stenosis, and tetralogy of Fallot; arrythmias, myocardial infarction, cardiomyopathy, pulmonary hypertension, and lung disease including but not limited to chronic bronchitis and emphysema. Symptoms of congestive heart failure include, but are not limited to, fatigue, breathing difficulty, pulmonary edema, and swelling of the ankles and legs.

As used herein, “Stunned myocardium” means heart muscle that is not functioning (pumping/beating) due to cardiac ischemia (lack of blood flow/oxygen to the vessels supplying the heat muscle).

As used herein, “Diastolic dysfunction” means an inability of the heart to fill with blood during diastole (the resting phase of heart contraction). This condition usually occurs in the setting of left ventricular hypertrophy. The heart muscle becomes enlarged and stiff such that it cannot fill adequately. Diastolic dysfunction can result in heart failure and inadequate heart function.

As used herein, “Pulmonary hypertension” means a disorder in which the blood pressure in the arteries supplying the lungs is abnormally high. Causes include, but are not limited to, inadequate supply of oxygen to the lungs, such as in chronic bronchitis and emphysema; pulmonary embolism, and intestinal pulmonary fibrosis. Symptoms and signs of pulmonary hypertension are often subtle and nonspecific. In the later stages, pulmonary hypertension leads to right heart failure that is associated with liver enlargement, enlargement of veins in the neck and generalized edema.

In a further embodiment of the methods of the invention, the methods are used for treating a heart muscle disorder comprising administering to an individual suffering from one or more of bradyarrythmia, bradycardia, congestive heart failure, stunned myocardium, pulmonary hypertension, and diastolic dysfunction, an amount effective to increase heart muscle contractile rate of an HSP20 composition according to the present invention.

Treating bradyarrythmia includes one or more of the following (a) improving the rate of the heartbeat to closer to normal levels for the individual, closer to a desired rate, or increasing to at least above 60 beats per minute; (b) limiting the occurrence of one or more of loss of energy, weakness, syncope, and hypotension in patients suffering from bradyarrythmia; (c) inhibiting worsening of one or more of loss of energy, weakness, syncope, and hypotension in patients suffering from bradyarrythmia and its symptoms; (d) limiting recurrence of bradyarrythmia in patients that previously suffered from bradyarrythmia; and (e) limiting recurrence of one or more of loss of energy, weakness, syncope, and hypotension in patients that previously suffered from bradyarrythmia.

Similarly, treating congestive heart failure includes one or more of the following (a) improving the heart's ability to pump adequate supplies of blood throughout the body to closer to normal levels for the individual, or closer to a desired pumping capacity; (b) limiting development of one or more of fatigue, breathing difficulty, pulmonary edema, and swelling of the ankles and legs in patients suffering from congestive heart failure; (c) inhibiting worsening of one or more of fatigue, breathing difficulty, pulmonary edema, and swelling of the ankles and legs in patients suffering from congestive heart failure and its symptoms; (d) limiting recurrence of congestive heart failure in patients that previously suffered from congestive heart failure; and (e) limiting recurrence of one or more of fatigue, breathing difficulty, pulmonary edema, and swelling of the ankles and legs in patients that previously suffered from congestive heart failure.

Treating stunned myocardium means one or more of (a) improving the ability of the heart muscle to pump by improving the oxygenation of the ischemic muscle, or by decreasing the need of the myocardial cells for oxygen and (b) limiting recurrence of stunned myocardium in patients that previously suffered from stunned myocardium.

Similarly, treating diastolic dysfunction includes one or more of (a) limiting heart failure and/or inadequate heart function by allowing the heart to relax and fill more completely; (b) limiting recurrence of diastolic dysfunction in patients that previously suffered from diastolic dysfunction; and (c) limiting recurrence of heart failure and/or inadequate heart function in patients that previously suffered from diastolic dysfunction.

Treating pulmonary hypertension includes one or more of the following (a) decreasing blood pressure in the arteries supplying the lungs to closer to normal levels for the individual, or closer to a desired pressure; (b) limiting the occurrence of one or more of enlargement of veins in the neck, enlargement of the liver, and generalized edema in patients suffering from pulmonary hypertension; (c) inhibiting worsening of one or more of enlargement of veins in the neck, enlargement of the liver, and generalized edema in patients suffering from pulmonary hypertension and its symptoms; (d) limiting recurrence of pulmonary hypertension in patients that previously suffered from pulmonary hypertension; and (e) limiting recurrence of one or more of enlargement of veins in the neck, enlargement of the liver, and generalized edema in patients that previously suffered from pulmonary hypertension.

In a further aspect, the present invention provides methods for inhibiting a heart muscle disorder comprising administering to an individual at risk of developing bradyarrythmia, bradycardia, congestive heart failure, stunned myocardium, pulmonary hypertension, and diastolic dysfunction an amount effective to increase heart muscle contractile rate of an HSP20 composition according to the present invention.

For example, methods to inhibit congestive heart failure involve administration of an HSP20 composition according to the present invention to a subject that suffers from one or more of hypertension, anemia, hyperthyroidism, heart valve defects including but not limited to aortic stenosis, aortic insufficiency, and tricuspid insufficiency; congenital heart defects including but not limited to coarctation of the aorta, septal defects, pulmonary stenosis, and tetralogy of Fallot; arrythmias, myocardial infarction, cardiomyopathy, pulmonary hypertension, and lung disease including but not limited to chronic bronchitis and emphysema.

Similarly, methods to inhibit bradyarrythmia involve administration of an HSP20 composition according to the present invention to a subject that suffer from one or more of coronary heart disease and atheroma formation, or that previously had a myocardial infarction or conduction disorder.

Similarly, methods to inhibit pulmonary hypertension involve administration of an HSP20 composition according to the present invention to a subject that suffers from one or more of chronic bronchitis, emphysema, pulmonary embolism, and intestinal pulmonary fibrosis.

Inhibiting stunned myocardium involves administration of an HSP20 composition according to the present invention to a subject that suffers from cardiac ischemia.

Treating diastolic dysfunction involves administration of an HSP20 composition according to the present invention to a subject that suffers from left ventricular hypertrophy

In a further embodiment of the methods of the invention, the method is used to promote wound healing and/or reduce scar formation. In these embodiments, an “individual in need thereof” is an individual that has suffered or will suffer (for example, via a surgical procedure) a wound that may result in scar formation, or has resulted in scar formation. As used herein, the term “wound” refers broadly to injuries to the skin and subcutaneous tissue. Such wounds include, but are not limited to lacerations; burns; punctures; pressure sores; bed sores; canker sores; trauma, bites; fistulas; ulcers; lesions caused by infections; periodontal wounds; endodontic wounds; burning mouth syndrome; laparotomy wounds; surgical wounds; incisional wounds; contractures after burns; tissue fibrosis, including but not limited to idiopathic pulmonary fibrosis, hepatic fibrosis, renal fibrosis, retroperitoneal fibrosis, cystic fibrosis, blood vessel fibrosis, heart tissue fibrosis; and wounds resulting from cosmetic surgical procedures. As used herein, the phrase “reducing scar formation” means any decrease in scar formation that provides a therapeutic or cosmetic benefit to the patient. Such a therapeutic or cosmetic benefit can be achieved, for example, by decreasing the size and/or depth of a scar relative to scar formation in the absence of treatment with the methods of the invention, or by reducing the size of an existing scar. As used herein, such scars include but are not limited to keloids; hypertrophic scars; and adhesion formation between organ surfaces, including but not limited to those occurring as a result of surgery. Such methods for reducing scar formation, are clinically useful for treating all types of wounds to reduce scar formation, both for reducing initial scar formation, and for therapeutic treatment of existing scars (i.e.: cutting out the scar after its formation, treating it with the compounds of the invention, and letting the scar heal more slowly). Such wounds are as described above. As used herein, the phrase “promoting wound healing” means any increase in wound healing that provides a therapeutic or cosmetic benefit to the patient. Such a therapeutic benefit can be achieved, for example, by one or more of increasing the rate of wound healing and/or increasing the degree of wound healing relative to an untreated individual. Such wounds are as described above.

In this embodiment, it is preferred that an HSP20 composition is disposed on or in a wound dressing or other topical administration. Such wound dressings can be any used in the art, including but not limited to films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), cellophane, and biological polymers such as those described in US patent application publication number 20030190364, published Oct. 9, 2003.

As used herein for all of the methods of the invention, an “amount effective” of an HSP20 composition is an amount that is sufficient to provide the intended benefit of treatment. An effective amount of an HSP20 composition that can be employed ranges generally between about 0.01 μg/kg body weight and about 10 mg/kg body weight, preferably ranging between about 0.05 μg/kg and about 5 mg/kg body weight. However dosage levels are based on a variety of factors, including the type of injury, the age, weight, sex, medical condition of the individual, the severity of the condition, the route of administration, and the particular compound employed. Thus, the dosage regimen may vary widely, but can be determined by a physician using standard methods.

The delivery of macromolecules such as peptides across the skin barrier is difficult due to the highly functionalized structure of the stratum corneum. Several compounds and techniques have been used to increase transportation of cargoes (macromolecules including drugs and peptides) across the skin barrier. These compounds and techniques include penetration enhancers such as oleic acid, drug delivery systems such as transferosomes, and physical techniques such as electroporation and iontophoresis and have been known to produce synergistic effects. Despite the benefits of these techniques and systems, topical and transdermal delivery of cargoes in therapeutics remains difficult. These difficulties are associated with skin toxicity of chemical enhancers at high concentrations, inconvenience of using electrical apparatuses at home, and high production costs of sophisticated drug delivery systems. There has been difficulty inducing skin and percutaneous penetration in PTDs linked to high molecular weight cargo peptide. Until recently this cargo has been only covalently linked to the PTD. It would therefore be desirable to have a PTD able to covalently or non-covalently attach to a variety of cargo with a broad range of molecular weights that would increase skin penetrations and percutaneous delivery of said cargo.

Thus, in a ninth aspect, the present invention provides methods for topical or transdermal delivery of an active cargo, comprising combining a transduction domain and an active cargo, and contacting the skin of a subject to whom the active agent is to be delivered, wherein the active cargo is delivered through the skin of the subject. In a preferred embodiment, the cargo is not covalently bound to the transduction domain. Exemplary cargo are as disclosed above. Details of this aspect are provided in the examples that follow. Examples of transduction domains that can be used according to this method of the invention include, but are not limited to the polypeptides of the present invention, as well as polypeptides comprising or consisting of one or more of the following:

(SEQ ID NO: 40)

(R)₄₋₉;

(SEQ ID NO: 18)

GRKKRRQRRRPPQ;

(SEQ ID NO: 19)

YARAAARQARA;

(SEQ ID NO: 20)

DAATATRGRSAASRPTERPRAPARSASRPRRPVE;

(SEQ ID NO: 21)

GWTLNSAGYLLGLINLKALAALAKKIL;

(SEQ ID NO: 22)

PLSSIFSRIGDP;

(SEQ ID NO: 23)

AAVALLPAVLLALLAP;

(SEQ ID NO: 24)

AAVLLPVLLAAP;

(SEQ ID NO: 25)

VTVLALGALAGVGVG;

(SEQ ID NO: 26)

GALFLGWLGAAGSTMGAWSQP;

(SEQ ID NO: 27)

GWTLNSAGYLLGLINLKALAALAKKIL;

(SEQ ID NO: 28)

KLALKLALKALKAALKLA;

(SEQ ID NO: 29)

KETWWETWWTEWSQPKKKRKV;

(SEQ ID NO: 30)

KAFAKLAARLYRKAGC;

(SEQ ID NO: 31)

KAFAKLAARLYRAAGC;

(SEQ ID NO: 32

AAFAKLAARLYRKAGC;

(SEQ ID NO: 33)

KAPAALAARLYRKAGC;

(SEQ ID NO: 34)

KAFAKLAAQLYRKAGC;

(SEQ ID NO: 35)

GGGGYGRKKRRQRRR;
and

(SEQ ID NO: 36)

YGRKKRRQRRR.

The present invention may be better understood with reference to the accompanying examples that are intended for purposes of illustration only and should not be construed to limit the scope of the invention, as defined by the claims appended hereto.

### Examples

### Example 1

FITC-(b)AWLRRIKA (SEQ ID NO: 37)(WLRRIKA (SEQ ID NO: 3) monomer), FITC-(b)AWLRRIKAWLRRIKA (SEQ ID NO: 38)(WLRRIKA (SEQ ID NO: 3) dimer), and FITC-(b)AWLRRIKAWLRRIKAWLRRIKA (SEQ ID NO: 39)(WLRRIKA (SEQ ID NO: 3) trimer) were synthesized on a 0.2 mmol scale using Fmoc-based solid phase peptide synthesis. The peptides were solubilized in water to create 3 mM stock solutions. 3T3 fibroblasts, cultured in Dulbecco's Modified Eagle Medium (DMEM) with 2 mM glutamine, pen/strep antibiotic, and 10% fetal bovine serum (FBS), were seeded at a density of 50,000 cells per well (1 ml of 50,000 cells/ml) in 4-well chambered slides (4 slides were used). The slides were incubated at 37° C. with 5% CO₂ in a humidified incubator for 4 hours to allow the cells to adhere to the slides. After 4 hours, each well was washed 3 times with phosphate buffered saline (PBS). 50 μl of a 3 mM stock of each peptide as well as a 3 mM stock of fluorescein in water were added to 15 ml tubes containing 3 ml of DMEM with 2 mM glutamine, antibiotic, and 10% FBS and to 15 ml tubes containing 3 ml of serum-free DMEM with 2 mM glutamine and antibiotic. Treatments were performed in duplicate for both serum-containing and serum-free DMEM. On each slide system, each well received media (either with or without serum) with fluorescein, WLRRIKA (SEQ ID NO: 3) monomer, WLRRIKA (SEQ ID NO: 3) dimer, or WLRRIKA (SEQ ID NO: 3) trimer. In all cases, the final peptide or fluorescein concentration was 50 μM per well. Once the treatments were added, the slides were incubated at 37° C. with 5% CO₂ in a humidified incubator for 1 hour. Then, each well was washed with PBS 3 times. Following the PBS wash, 0.2 ml trypsin was added to each well to digest residual peptide bound to the outer cellular membranes, and the slides were incubated at 37° C. for 10 minutes. To inactivate the trypsin, 1 ml DMEM with serum was added to each well, and the slides were incubated for 4 hours to allow cells to reattach to the slides. After 4 hours, the cells were washed 3 times in PBS, and 1 ml of DMEM with serum was added to each well. Then, the slides were imaged using a 40× objective. Phase and fluorescent images were acquired with 75 ms exposure times. No fluorescent signal was observed for any condition wherein cells were treated with fluorescein. Thus, the fluorescein treatment acted as a negative control. A lack of fluorescence for cells treated with WLRRIKA (SEQ ID NO: 3) monomer regardless of whether or not the media contained serum indicated that the WLRRIKA (SEQ ID NO: 3) monomer, by itself, was not able to penetrate the cells. The WLRRIKA (SEQ ID NO: 3) dimer and WLRRIKA (SEQ ID NO: 3) trimer treatments resulted in bright fluorescence within the cells, regardless of whether the cells were incubated with or without serum.

### Example 2

Peptides were designed to test their ability to carry molecules across cell membranes and the skin. Peptide W3 (WLRRIKAWLRRIKAWLRRIKA) (SEQ ID NO: 5) is a trimer of peptide WLRRIKA (SEQ ID NO: 3). The molecule (“cargo”) that was chosen to be carried across the skin was a fragment of HSP20 (WLRRApSAPLPGLK, where pS is phosphoserine) (SEQ ID NO: 9) linked to a fluorescent probe (fluorescein isothiocyanate, FITC). The controls were known protein transduction domains, TAT (YGRKKRRQRRR)(SEQ ID NO: 36) and PTD (YARAAARQARA)(SEQ ID NO: 19).

The peptides were synthesized at Arizona State University (ASU) using an Automated Peptide Synthesizer (Apex 396, Advanced ChemTech, Louisville, Ky.), and solid phase technique. FITC-labeled peptides were obtained by linking FITC to β-alanine added to the N-terminus of the peptide. The peptides were purified by FPLC (Akta Explorer, Amersham Pharmacia Biotech, Piscataway, N.J.) using a reversed-phase column, and identified by MADI-TOF or ESI-MS (Waters Corporation, Milford, Mass.).

The in vitro model used to assess transduction across cell membranes was primary rat astrocyte cells. Cells were isolated as described (Innocenti et. al., J. Neurosci. 20:1800-1808, 2000), seeded at ˜3×10⁴ cells/cm², and cultured in full serum (10% FBS in α-MEM) overnight. Some cells were serum starved by culturing in 0.5% FBS for 1-24 hours prior to treatment with transduction peptides. Cells treated with 50 μM W3 (WLRRIKA (SEQ ID NO: 3) trimer) for 1 h to demonstrate efficient transduction that persists at least 24 hours. There was a dose dependency for transduction using the pseudo-dimer WLRRIKA-WLRRApSAPLPGLK (SEQ ID NO: 10)(WL-P20), with efficient transduction at 50 μM, slightly reduced transduction at 25 μM, and no transduction at 1 μM. As a control, the pseudo-monomer WLRRIKA-(WL-scrP20) (SEQ ID NO: 3) was tested at 50, 25, and 1 μM, but no transduction was observed.

Maximal transduction occurred at approximately 20 minutes for the pseudo-dimer (WL-P20) (SEQ ID NO: 10); similar results were obtained using W3.

The ex vivo model selected for skin transduction studies was freshly excised porcine ears. After obtaining ear from a local abattoir, the skin from the outer surface of the ear was carefully dissected (making sure that the subcutaneous fat was maximally removed) as previously described. The cleaned porcine ear skin was immediately mounted in a modified Franz diffusion cell (diffusion area of 1 cm²; Laboratory Glass Apparatus, Inc, Berkeley, Calif.), with the stratum corneum facing the donor compartment (where the formulation was applied) and the dermis facing the receptor compartment, which was filled with PBS (3 mL). The system was maintained at 37° C. and under constant stirring. PBS solutions or propylene glycol formulations of the peptides (70 μl) were applied in the donor compartment of the diffusion cell for up to 8 hours.

At the end of the experiment, skin surfaces were thoroughly washed with distilled water to remove excess formulation. To separate the stratum corneum (SC) from the remaining epidermis (E) and dermis (D), skin pieces were subjected to tape stripping. The skin was stripped with 15 pieces of adhesive tape, and the tapes containing the SC were immersed in 3 mL of a water:methanol (1:1 v/v) solution vortexed for 2 minutes and bath sonicated for 30 minutes. The remaining [E+D] was cut in small pieces, vortexed for 2 minutes in 2 mL of a water:methanol (1:1 v/v) solution, and homogenized using a tissue homogenizer for 1 minute and bath sonication for 30 minutes. The resulting mixture was then centrifuged for 1 minute. The amount of peptides that permeated across the skin was determined in the receptor phase; 1.5 mL of the receptor phase was withdrawn, lyophilized and the residue was suspended in 150 μL of water.

The amount of FITC-labeled peptides that penetrated into SC and [E+D], and permeated across the skin was spectrofluorimetrically determined using a Gemini SpectraMax™ platereader (Molecular Devices, Sunnyvale, Calif.) with excitation at 495 nm and emission at 518 nm. Standard curves of the peptides were used as reference.

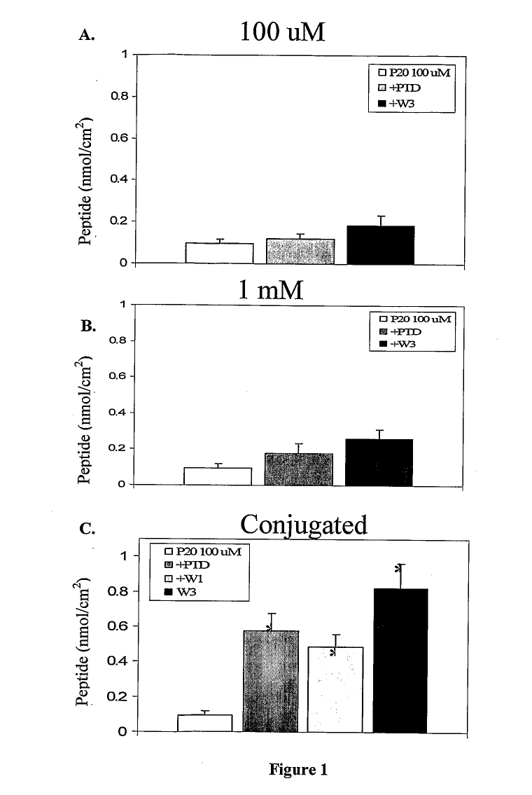

Concentrations of 100 uM of PTD or W³ (non-covalently bound) did not carry P20 (SEQ ID NO: 9) across the skin (FIG. 1, Panel A). However, there was significant skin penetration [E+D] when 1 mM W3 was used to carry P20 (SEQ ID NO: 9) (FIG. 1, Panel B). The skin penetration was significantly enhanced when P20 (SEQ ID NO: 9) was conjugated to PTD or W1 or when W3 was used alone, indicating that conjugated transduction domains result in enhanced skin penetration (100 uM, FIG. 1, Panel C). This is the first evidence that we are aware of that a protein transduction domain (W3) can carry noncovalently linked molecules into the skin. These data have significant implications for delivery of biologically active molecules into the skin for therapeutic purposes.

### Example 3

### Protein Transduction Domain Penetration of Skin

Methods: YARA (defined as YARAAARQARA (SEQ ID NO: 19), TAT (SEQ ID NO: 43), YKAc (defined as YKALRISRKLAK (SEQ ID NO: 41)), P20 (defined as WLRRASAPLPGLK (SEQ ID NO: 9)), YARA-P20 (defined as YARAAARQARAWLRRASAPLPGLK (SEQ ID NO: 42), and TAT-P20 (defined as YGRKKRRQRRRWLRRASAPLPGLK (SEQ ID NO: 43) were synthesized by Fmoc chemistry. Porcine ear skin mounted in a Franz diffusion cell was used to assess the topical and transdermal delivery of fluorescently tagged peptides in the presence or absence of lipid penetration enhancers (monoolein or oleic acid). The peptide concentrations in the skin (topical delivery) and receptor phase (transdermal delivery) were assessed by spectrofluorimetry. Fluorescence microscopy was used to visualize the peptides in different skin layers.

Results: YARA (SEQ ID NO: 19) and TAT (SEQ ID NO: 43), but not YKAc (SEQ ID NO: 41), penetrated abundantly in the skin and permeated modestly across this tissue. Monoolein and oleic acid did not enhance the topical and transdermal delivery of TAT (SEQ ID NO: 43) or YARA (SEQ ID NO: 19), but increased the topical delivery of YKAc (SEQ ID NO: 41). Importantly, YARA (SEQ ID NO: 19) and TAT (SEQ ID NO: 43) carried a conjugated peptide, P20 (SEQ ID NO: 9) into the skin, but the transdermal delivery was very small. Fluorescence microscopy confirmed that free and conjugated PTDs reached viable layers of the skin.

Conclusions: YARA (SEQ ID NO: 19) and TAT (SEQ ID NO: 43) penetrate in the porcine ear skin in vitro and carry a conjugated model peptide, P20 (SEQ ID NO: 9), with them. Thus, the use of PTDs can be a useful strategy to increase topical delivery of peptides for treatment of cutaneous diseases.

The first aim of the present study was to evaluate the ability of YARA (SEQ ID NO: 19) to penetrate in the skin in vitro. The penetration of YARA (SEQ ID NO: 19) was compared to that of the well-known transduction domain TAT (SEQ ID NO: 43), and of the nontransducing peptide, YKALRISRKLAK (SEQ ID NO: 41) (YKAc); all peptides have similar molecular weight.

Our second aim was to examine the influence of chemical penetration enhancers (monoolein and oleic acid) on the topical and transdermal delivery of YARA (SEQ ID NO: 19), TAT (SEQ ID NO: 43), and YKAc (SEQ ID NO: 41). Our third aim was to verify the ability of YARA (SEQ ID NO: 19) and TAT (SEQ ID NO: 43) to increase the skin penetration and percutaneous delivery of a conjugated model peptide, P20 (SEQ ID NO: 9). This peptide is hydrophilic and has a high molecular weight (2005 Da). Many peptides with similar characteristics have therapeutic potential for treatment of skin diseases (6,31), and their skin penetration has been shown to be extremely poor (32).

### Materials and Methods

Materials: Reagents for peptide synthesis, including amino acids, were purchased from Advanced ChemTech (Louisville, Ky., USA), Anaspec (San Jose, Calif., USA), Applied Biosystems (Foster City, Calif., USA), and Novobiochem (San Diego, Calif., USA). Fluorescein-5-isothiocyanate (FITC ‘Isomer 1’) was purchased from Molecular Probes (Eugene, Oreg., USA). Monoolein was obtained from Quest (Naarden, The Netherlands) and oleic acid from Sigma (St. Louis, Mo., USA). All solvents and chemicals were of analytical grade. Freshly excised porcine ears were obtained from a local abattoir (Southwest meat processing, Queen Creek, Ariz., USA)

Peptide synthesis: Fluorescein isothiocyanate (FITC)-labeled peptides, including YARA (YARAAARQARA, MW: 1668)(SEQ ID NO: 19), TAT (YGRKKRRQRRR, MW: 2020)(SEQ ID NO: 36), YKAc (YKALRISRKLAK, MW: 1907)(SEQ ID NO: 41), P20 (WLRRASAPLPGLK, MW: 2005)(SEQ ID NO: 9), YARA-P20 (YARAAARQARAWLRRASAPLPGLK, MW: 3111)(SEQ ID NO: 42), and TAT-P20 (YGRKKRRQRRRWLRRASAPLPGLK, MW: 3466)(SEQ ID NO: 43) were synthesized using an Automated Peptide Synthesizer (Apex 396, Advanced ChemTech, Louisville, Ky., USA) and solid phase technique. FITC was linked to a β-alanine residue added to the N-terminus of the peptide. The peptides were purified by Fast Protein Liquid Chromatography (FPLC, Akta Explorer, Amersham Pharmacia Biotech, Piscataway, N.J., USA) using a reversed-phase column and identified by Matrix Assisted Laser Desorption-Ionization Time-of-Flight Mass Spectrometry (MALDI-TOF-MS, Applied Biosystems, Foster City, Calif., USA) or Electrospray Ionization Mass Spectrometry (ESI-MS, Waters Corporation, Milford, Mass., USA).

Formulations: Except in the experiments involving chemical penetration enhancers, FITC-labeled peptides were dissolved in phosphate-buffered saline (PBS, 10 mM, pH 7.2); the peptide concentration was 100 μM. In the experiment involving penetration enhancers, PBS could not be used as a solvent because of the lipophilic nature of monoolein and oleic acid. Propylene glycol was used as a solvent, since it solubilizes both lipids and peptides. Formulations of FITC-TAT (SEQ ID NO: 43), FITC-YARA (SEQ ID NO: 19), and FITC-YKAc (SEQ ID NO: 41) (100 μM) in propylene glycol containing 10% (w/w) monoolein, 5% (w/w) oleic acid, or none of these penetration enhancers were prepared. The formulations were prepared by mixing monoolein or oleic acid with propylene glycol and adding the peptides to the system immediately thereafter.

In vitro skin penetration: To evaluate the topical and transdermal delivery of the peptides, we applied the formulations of FITC-labeled TAT (SEQ ID NO: 43), YARA (SEQ ID NO: 19), YKAc (SEQ ID NO: 41), P20 (SEQ ID NO: 9), YARA-P20 (SEQ ID NO: 42), or TAT-P20 (SEQ ID NO: 43) on the surface of freshly excised porcine ear skin mounted in a Franz diffusion cell.

Porcine ear skin was used as model skin for in vitro skin penetration studies because of its similarity with human skin, especially regarding histological and biochemical properties and permeability to drugs (33). Freshly excised porcine ears were obtained from a local abattoir. The skin from the outer surface of the ear was carefully dissected; making sure that the subcutaneous fat was maximally removed (34). Maximum care was taken to maintain the integrity of the skin, which was assured by histology. The cleaned porcine ear skin was immediately mounted in a Franz diffusion cell (diffusion area of 1 cm²; Laboratory Glass Apparatus, Inc, Berkeley, Calif., USA), with the stratum corneum facing the donor compartment (where the formulation was applied) and the dermis facing the receptor compartment, which was filled with PBS (100 mM, pH 7.2, 3 mL). The receptor phase was maintained at 37° C. and under constant stirring. To achieve higher reproducibility, the skin samples were equilibrated to the diffusion cell conditions for 30 minutes before application of any formulation.

PBS solutions or propylene glycol formulations of the peptides (70 μL each) were applied to the skin surface (donor compartment). The concentration of FITC-YARA (SEQ ID NO: 19), FITC-TAT (SEQ ID NO: 43), and FITC-YKAc (SEQ ID NO: 41) in the skin (an indicator of topical delivery) and receptor phase (an indicator of transdermal delivery) was determined at 4 h post-application. The concentrations of FITC-P20 (SEQ ID NO: 9), FITC-YARA-P20 (SEQ ID NO: 42), and FITC-TAT-P20 (SEQ ID NO: 43) in the skin and receptor phase were determined at 0.5, 1, 2, 4, and 8 hour post-application.

At the end of the experiment, skin surfaces were thoroughly washed with distilled water to remove excess formulation and carefully wiped with a tissue paper. To separate the stratum corneum (SC) from the remaining epidermis (E) and dermis (D), the skin was subjected to tape stripping. The skin was stripped with 15 pieces of adhesive tape (3M, St. Paul, Minn., USA), and the tapes containing the SC were immersed in 3 mL of a water:methanol (1:1 v/v) solution, vortexed for 2 min, and bath sonicated for 30 minutes. The remaining [E+D] was cut in small pieces, vortexed for 2 minutes in 2 mL of a water:methanol (1:1 v/v) solution, and homogenized using a tissue grinder for 1 minute and bath sonication for 30 minutes. The resulting mixture was centrifuged for 1 minute. The peptide present in the receptor phase was concentrated (10×) as follows. Samples (2 mL) of the receptor phase were lyophilized for 24 hours, and the residue was dissolved in 200 μL of a hidroalcoholic (20% of ethanol) solution.

All solutions were subjected to fluorimetry analysis using a Gemini SpectraMax™ platereader (Molecular Devices, Sunnyvale, Calif., USA) with excitation at 495 nm and emission at 518 nm. The method was linear within the concentration range studied (0.05-2.0 μM). To evaluate the recovery of the peptides from the skin in the extraction procedure, tissues sections (1 cm²) were spiked with 20 μL of 0.2 and 0.5 mM solutions of the peptides. The skin sections were homogenized using a tissue grinder, vortex-mixer, and bath sonicator, as described above. The recovery of the peptides was 83-90%, depending on the peptide. We accounted for such a recovery percentage in the quantification of peptides.

Histology: At 4 hours post-application of FITC-labeled YARA (SEQ ID NO: 19), TAT (SEQ ID NO: 36), YKAc (SEQ ID NO: 41), P20 (SEQ ID NO: 9), YARA-P20 (SEQ ID NO: 42), and TAT-P20 (SEQ ID NO: 43), the diffusion area of skin samples were frozen using isopentane at −30° C., embedded in Tissue-Tek® OCT compound (Pelco International, Redding, Calif., USA), and sectioned using a cryostat microtome (Leica, Wetzlar, Germany). The skin sections (8 μm) were mounted on glass slides. The slides were visualized without any additional staining or treatment through a 20× objective using a Zeiss microscope (Carl Zeiss, Thornwood, N.Y., USA) equipped with a filter for FITC and AxioVision software.

Statistical analysis. The results are reported as means±SD. Data were statistically analyzed by nonparametric Kruskal-Wallis test followed by Dunn post-test (6). The level of significance was set at p<0.05.

### Results

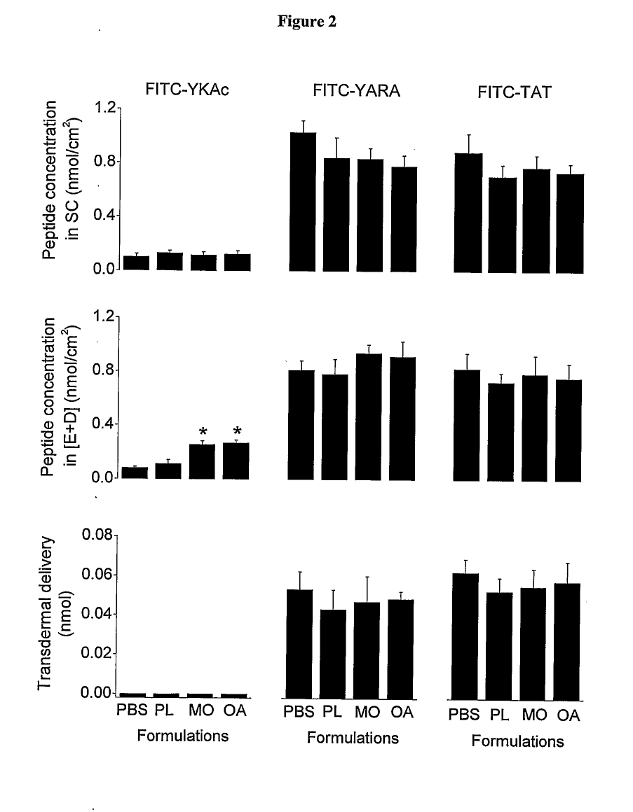

Topical and transdermal delivery of PTDs: Our first aim was to evaluate the ability of PTDs to penetrate in the skin and permeate across this tissue, so that they could be used as carrier for topical and/or transdermal delivery of peptides (results shown in FIG. 2). In this experiment, PBS was used as vehicle. We determined the penetration of FITC-YARA (SEQ ID NO: 19), FITC-TAT (SEQ ID NO: 43), and FITC-YKAc (SEQ ID NO: 41) in the SC and [E+D] as well as their transdermal delivery at 4 hours post-application. The penetration of the control, nontransducing peptide FITC-YKAc (SEQ ID NO: 41) in both SC and [E+D] was very small, and no peptide was found in the receptor phase (indicating no transdermal delivery). On the other hand, the penetration of FITC-YARA (SEQ ID NO: 19) and FITC-TAT (SEQ ID NO: 43) in the SC and [E+D] was 8-10 times higher than that of the control peptide. The transdermal delivery of FITC-YARA (SEQ ID NO: 19) and FITC-TAT (SEQ ID NO: 43) was small at 4 hours post-application, and there was no significant difference in the amount of FITC-YARA (SEQ ID NO: 19) detected in the receptor phase compared to FITC-TAT (SEQ ID NO: 43). Only 0.053±0.009 nmol of FITC-YARA (SEQ ID NO: 19) and 0.058±0.008 nmol of FITC-TAT (SEQ ID NO: 43) were found in the receptor phase, which means that the amount of YARA (SEQ ID NO: 19) and TAT (SEQ ID NO: 43) that penetrated into the skin (SC+[E+D]) was respectively 34 and 30 times higher than the amount that permeated across the skin.

Influence of penetration enhancers on topical and transdermal delivery of PTDs: We next evaluated the influence of monoolein and oleic acid on the topical and transdermal delivery of FITC-labeled YARA (SEQ ID NO: 19), TAT (SEQ ID NO: 43), and YKAc (SEQ ID NO: 41) (results shown in FIG. 2). In this experiment, the permeation enhancers and peptides were dissolved in propylene glycol. Compared to PBS, propylene glycol did not influence the skin penetration of the YKAc (SEQ ID NO: 41), YARA (SEQ ID NO: 19), and TAT (SEQ ID NO: 43) at 4 hours post-application. Notably, addition of monoolein or oleic acid to the formulations significantly (p<0.05) increased (˜2.5 times) the penetration of the nontransducing peptide, FITC-YKAc (SEQ ID NO: 41), in [E+D]. The same penetration enhancers, however, failed to further increase the already high topical or the transdermal delivery of TAT (SEQ ID NO: 43) and YARA (SEQ ID NO: 19).

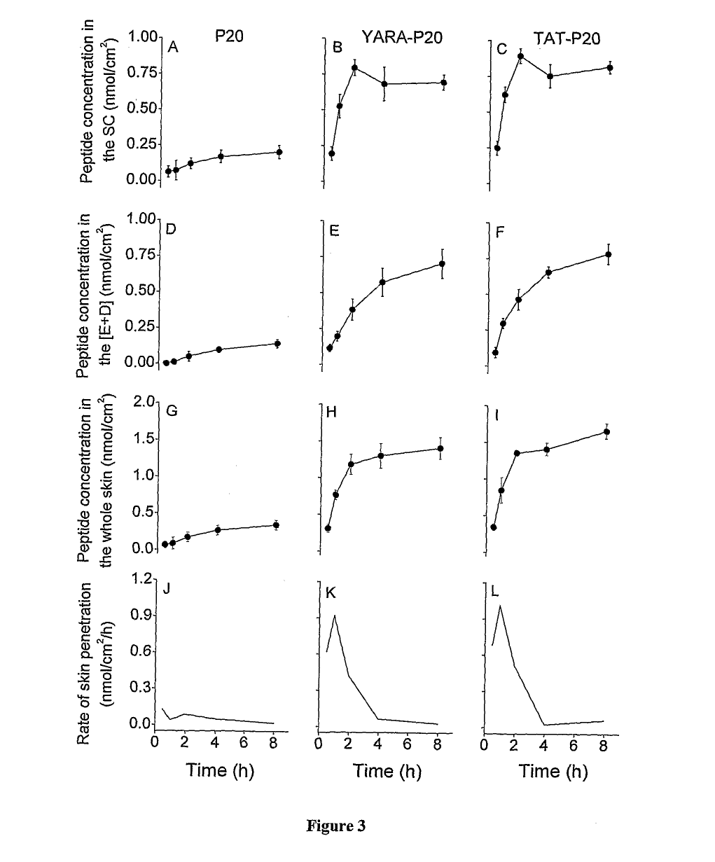

Transport of the conjugated peptide P20 (SEQ ID NO: 9) into and across the skin by PTDs: Having demonstrated that FITC-YARA (SEQ ID NO: 19) penetrates in the skin in a similar extent to FITC-TAT (SEQ ID NO: 43), we evaluated its ability to increase the penetration of a conjugated peptide. We attached the peptide P20 (SEQ ID NO: 9) to FITC-YARA (SEQ ID NO: 19) and FITC-TAT (SEQ ID NO: 43), and evaluated their topical and transdermal delivery as a function of time. The PTDs studied were able to carry conjugated P20 (SEQ ID NO: 9) into SC and [E+D] (FIG. 3). When P20 (SEQ ID NO: 9) was conjugated to YARA (SEQ ID NO: 19) and TAT (SEQ ID NO: 43), its penetration in both SC and [E+D] was significantly higher (p<0.05) than that of nonconjugated P20 (SEQ ID NO: 9) at all time points studied (FIGS. 3A-F). The concentration of YARA-P20 (SEQ ID NO: 42) and TAT-P20 (SEQ ID NO: 43) in [E+D] was progressively increased (p<0.05) from 0.5 to 4 hours post-application (FIGS. 3E and 3F), but no further increase was found between 4 and 8 hours. The concentration of the PTD-P20 conjugates in the viable layers of skin ([E+D]) was 5 to 7 times higher than that of nonconjugated P20 (SEQ ID NO: 9) at 4 and 8 hours post-application. The maximal rate of penetration of YARA-P20 (SEQ ID NO: 42) and TAT-P20 (SEQ ID NO: 43) in the whole skin (SC+[E+D]) was achieved at 1 h post-application (FIGS. 3K-L). The transdermal delivery of FITC-YARA-P20 (SEQ ID NO: 42) and FITC-TAT-P20 (SEQ ID NO: 43) was very small (0.031±0.011 nmol and 0.027±0.009 nmol for FITC-YARA-P20 (SEQ ID NO: 42) and FITC-TAT-P20 (SEQ ID NO: 43), respectively); the peptides were detected in the receptor phase only at 8 hours post-application. FITC-P20 did not permeate across the skin at all.

Visualization of the skin penetration of peptides using fluorescence microscopy: As expected, the skin treated with PBS presented a very weak auto-fluorescence (especially the SC). Treatment of the skin with FITC-YARA (SEQ ID NO: 19) and FITC-TAT (SEQ ID NO: 43) resulted in a strong fluorescent staining of SC and viable epidermis. Some fluorescence could also be observed in the dermis, demonstrating that these PTDs were able to cross the SC and reach the viable layers of the skin. On the other hand, FITC-YKAc (SEQ ID NO: 41) was predominantly localized in the SC, and only a very weak fluorescence was observed in the epidermis. When the skin was treated with FITC-labeled YARA (SEQ ID NO: 19) or TAT (SEQ ID NO: 43) conjugated with P20 (SEQ ID NO: 9), we also observed the presence of strong fluorescence in the SC and viable epidermis. When the skin was treated with FITC-P20 (SEQ ID NO: 9), fluorescence was found only in the SC.

### Discussion

In the present study, we demonstrated the ability of the PTD YARA (SEQ ID NO: 19) to penetrate in the skin of porcine ears in vitro. Despite the fact that YARA (SEQ ID NO: 19) has previously been demonstrated to transduce into cells more effectively than TAT (SEQ ID NO: 43) in vitro and in vivo (28), we found no significant difference in the ability of these two peptides to penetrate in the skin. On the other hand, the skin penetration of a nontransducing peptide of similar molecular weight, YKAc (SEQ ID NO: 41), was negligible in both SC and [E+D], which is expected since this peptide is hydrophilic and has a high molecular weight (1907 Da).

The influence of chemical enhancers on the skin penetration of peptides was evaluated using propylene glycol formulations containing monoolein and oleic acid. The use of propylene glycol as a solvent had no influence on the topical and transdermal delivery of the peptides studied. Formulations containing monoolein or oleic acid did significantly enhance the penetration of the control, nontransducing peptide YKAc (SEQ ID NO: 41) in the skin. This observation is consistent with the fact that monoolein and oleic acid act by several mechanisms to increase the permeability of the SC to drugs, including peptides (36,37). These mechanisms include modification of lipid domains and extraction of lipids from the SC (10, 36-38). On the other hand, neither monoolein nor oleic acid influenced the topical and transdermal delivery of YARA (SEQ ID NO: 19) or TAT (SEQ ID NO: 43). The results suggest that the chemical penetration enhancers studied can be useful to increase the skin penetration of peptides, but only when these have no transduction ability and do not penetrate in the skin at a high extent by themselves.

The skin penetration of YARA-P20 (SEQ ID NO: 42) and TAT-P20 (SEQ ID NO: 43) was very fast, and the conjugates were able to penetrate in the SC and [E+D] to a higher extent compared to P20 (SEQ ID NO: 9) alone. Fluorescence microscopy analysis confirmed that YARA-P20 (SEQ ID NO: 42) and TAT-P20 (SEQ ID NO: 43) crossed the SC barrier efficiently, and revealed that these relatively large molecules were homogeneously distributed in viable epidermis. It has been shown that conjugates of PTDs-peptides can penetrate very fast in the mice skin, achieving high concentrations as fast as 1 hour post-application (39,40). Robbins et al. (39) observed only slight differences in the skin penetration of heptarginine-hemaglutinin epitope from 0.5 to 1 hour post-application. In the present study, we observed that the maximum rate of skin penetration of the conjugates occurred at 1 hour post-application, but their concentration in the skin progressively increased until 4 hour post-application (p<0.05). The skin penetration of protein transduction domains conjugated to peptides might vary depending on the PTD and the experimental model skin used, since the mechanism of penetration might vary among different compounds, and the properties and characteristics of the skin might differ among animals (20,33).

Although the metabolic activity in the skin is smaller than the activity in other tissues (such as mucosa), the stability of peptides in the skin is an important issue (41). Several authors have demonstrated that FITC-labeled macromolecules present good stability in biological tissues, including skin. The integrity of FITC-poly-lysine in the receptor phase of a diffusion cell was demonstrated by HPLC and mass spectrometry, even after the exposure of the compound to electrical current or ultrasound (42, 43). FITC-labeled dextrans of different molecular weight had their structure integrity maintained after transdermal delivery, as demonstrated by size-exclusion chromatography (44). Last but not least, the integrity of FITC-oligonucleotides in the skin was demonstrated by Western blot (45). The stability of the PTDs used in this study has also been demonstrated before after incubation at 37° C. for several hours in contact with biological tissues. The pharmacological activity of the conjugate YARA-P20 (SEQ ID NO: 42) was preserved after its incubation for 2 days at 37° C. with vein segments (29).

Thus, topical administration of conjugates of PTD-peptides may have therapeutic potential for local skin disorders. Topical delivery of peptides has been increasingly studied due to the importance of these compounds for the treatment of skin diseases and for the improvement of skin properties (in the case of cosmetics). Topical administration of several peptides would be attractive, including TGF-β, leptin (both for wound healing), INF-α (antiviral), cyclosporin (for treatment of auto-immune diseases), bacitracin (for skin infections), and palmytoyl-glycyl-hystidyl-lysine tripeptide (for stimulation of collagen synthesis), among many others (6,11,25,31,46-48). In addition, several peptides have been applied to the skin and studied as antigens for the development of topical vaccines (49). In this context, the use of PTDs could be useful for successfully increasing peptides delivery to the skin, a significant achievement that could bring therapeutic benefits associated with avoidance of systemic side effects and patient commodity.

Even though the skin penetration of different PTDs has been reported in the literature (25-27,39), the exact mechanism of action remains unknown. The intercellular lipid domain of the stratum corneum differs from cell membranes not only on lipid composition, but also on water content and lipid/protein ratio (50). In addition, the outermost layer of the skin is composed of non-viable cells, and endocytosis is not expected. Hence, the mechanism for PTDs to penetrate in the skin is likely different from that for them to cross cell membranes. Rothbard et al. (25) suggested that the SC is a metabolically active environment (although it is not constituted of viable cells), which can contribute to the transport of PTDs. Moreover, it is well known that several PTDs are able to interact with lipids (51), which may be important for their transport across the SC. Indeed, poly-L-arginine was demonstrated to increase the permeability of tight junctions of the nasal epithelium (52) and the transport of a dextran. This effect was triggered by interaction of poly-arginine with negatively charged lipids of the cell (53). The presence of tight junctions in the skin has already been demonstrated (54), and the disassembly of these structures by the PTDs studied might be important for their penetration into the viable layers of the skin. Moreover, PTDs might penetrate different layers of the skin, and the resulting gradient might be the force driving the penetration of PTDs in the skin (25).

Although the topical delivery of YARA-P20 (SEQ ID NO: 42) and TAT-P20 (SEQ ID NO: 43) was high, we found that their transdermal delivery was small and occurred somewhat slowly, at least in vitro. In vivo, however, the transdermal delivery of these compounds might be more substantial and faster since living skin is more dynamic than ex vivo skin used in these experiments, and further studies are necessary to evaluate whether topically administered PTD-P20 may produce effects in deeper tissues. This may be of special interest due to the recently demonstrated ability of P20 (SEQ ID NO: 9) to cause vasodilation (29,30). Such an effect may be, for example, used for the topical treatment of sexual dysfunction in males and/or females.

### Conclusions

We conclude that the PTDs YARA (SEQ ID NO: 19), TAT (SEQ ID NO: 43), and their conjugates with the peptide P20 (SEQ ID NO: 9) rapidly penetrate in porcine skin in vitro at a high extent. These results suggest that PTDs can be used as carrier molecules to deliver peptides of therapeutic interest to the skin. We also conclude that the skin penetration of YARA (SEQ ID NO: 19) and TAT (SEQ ID NO: 43) is not further improved by formulations containing the chemical penetration enhancer monoolein or oleic acid, even though the same penetration enhancers improve the topical delivery of a large, but nontransducing, peptide.

### BIBLIOGHRAPHY

1. M. R. Prausnitz, S. Mitragotri, R. Langer, Current status and future potential of transdermal drug delivery. Nat. Rev. Drug Discov. 3:115-124 (2004).
2. K. C. Madison. Barrier function of the skin: “La Raison d'etre” of the epidermis. J. Invest. Dermatol, 121:231-241 (2003).
3. B. Barry. Breaching the skin's barrier to drugs. Nat. Biotechnol. 22:165-167 (2004).
4. P. Karande, A. Jain, S. Mitragotri, Discovery of transdermal penetration enhancers by high-throughput screening, Nat. Biotechnol. 22:192-197 (2004).
5. G. Cevc, A. Schatzlein, G. Blume. Transdermal drug carriers: basic properties, optimizations and transfer efficiency in the case of epicutaneously applied peptides. J. Control. Rel. 36:3-16 (1995).
6. B. Godin, E. Touitou. Mechanism of bacitracin permeation enhancement through the skin and cellular membranes from an ethosomal carrier. J. Control. Rel. 94:365-379 (2004).
7. O. Pillai, V. Nair, R. Panchagnula. Transdermal iontophoresis of insulin: IV. Influence of chemical enhancers. Int. J. Pharm. 269:109-120 (2004).
8. Y. N. Kalia, A. Naik, J. Garrison, R. H. Guy. Iontophoretic drug delivery. Adv. Drug Deliv. Rev. 56:619-658 (2004).
9. S. Mitragotri. Synergistic effect of enhancers for transdermal drug delivery. Pharm. Res. 17:1354-1359 (2000).
10. H. D. C. Smyth, G. Becket, S. Mehta. Effect of permeation enhancer pretreatment on the iontophoresis of luteinizing hormone releasing hormone (LHRH) through human epidermal membrane (HEM). J. Pharm. Sci. 9:11296-1307 (2002).
11. R. R. Boinpally, S. L. Zhou, G. Devraj, P. K. Anne, S. Poondru, B. R. Jasti, Iontophoresis of lecithin vesicles of clyclosporin A. Int. J. Pharm. 274:185-190 (2004).
12. M. Lindgren, M. Hallbrink, A. Prochiantz, U. Langel. Cell-penetrating peptides. Trends Pharmacol. Sci. 21:99-103 (2000).
13. S. R. Schwarze, S. F. Dowdy. In vivo protein transduction: intracellular delivery of biologically active proteins, compounds and DNA. Trends Pharmacol. Sci. 21:45-48 (2000).
14. M. Lundberg, S. Wikstrom, M. Johansson. Cell surface adherence and endocytosis of protein transduction domains. Mol. Therapy 8:143-150 (2003).
15. E. L. Snyder, S. F. Dowdy. Cell penetrating peptides in drug delivery. Pharm. Res. 21:389-393 (2004).
16. J. Zaro, W. C. Shen. Quantitative comparison of membrane transduction and endocytosis of oligopeptides. Biom. Biophys. Res. Comm. 307:241-247 (2003).
17. S. R. Schwarze, A. Ho, A. Vocero Akbani, S. F. Dowdy. In vivo protein transduction: delivery of a biologically active protein into the mouse. Science 285:1569-1572 (1999).
18. C. R. Flynn, P. Komalavilas, D. Trssier, J. Thresher, E. E. Niederkofler, C. M. Dreiza, R. W. Nelson, A. Panitch, L. Joshi, C. M. Brophy. Transduction of biologically active motifs of the small heat shock related protein HSP20 leads to relaxation of vascular smooth muscle. Faseb J. 17:1358-1360 (2003).
19. V. P. Torchilin, T. S. Levchenko. TAT-liposomes: a novel intracellular drug carrier. Cur Prot. Pept. Sci. 4:133-140 (2003)
20. S. Console, C. Marty, C. García-Escheverría, R. Schwendener, K. Ballmer-Hefer. Antennapedia and HIV transactivator (TAT) “protein transaction domains” promote endocytosis of high molecular weight cargo upon binding to cell surface glycosaminoglycans. J. Biol. Chem. 278:35109-35114 (2003).
21. D. Derossi, S. Calvet, A. Trembleau, A. Brunissen, G. Chassaing, A. Prochiantz. Cell internalization of the third helix of Antennapedia homeodomain is receptor-independent. J. Biol. Chem. 271:18188-18193 (1996).
22. E. Vives, P. Brodin, B. Lebleu. A truncated HIV-1 Tat protein basic domain rapidly translocates through the plasma membrane and accumulates in the cell nucleus. J. Biol. Chem. 272:16010-16017 (1997).
23. J. P. Richard, K. Melikov, E. Vives, C. Ramos, B. Verbeure, M. J. Gait, L. V. Chernomordik, B. Lebleu. Cell penetrating peptides: a reevaluation of the mechanism of cellular uptake. J. Biol. Chem. 278:585-590 (2003).
24. P. E. G. Thoren, D. Persson, P. Isakson, M. Goksor, A. Onfelt, B. Norden. Uptake of analogs of penetratin, TAT (48-60) and oligoarginine in live cells. Biochem. Biophys. Res. Commun. 307:100-107 (2003).
25. J. B. Rothbard, S. Garlington, Q. Lin, T. Kirschberg, E. Kreider, P. L. Mcgrane, P. A. Wender, P. A. Khavari. Conjugation of arginine oligomers to cyclosporin A facilitates topical delivery and inhibition of inflammation. Nature Med. 6:1253-1257 (2000).
26. J. M. Lim, M. Y. Chang, S. G. Park, N. G. Kang, Y. S. Song, Y. H. Lee, Y. C. Yoo, W. G. Cho, S. Y. Choi, S. H. Kang. Penetration enhancement in mouse skin and lipolysis in adipocytes by TAT-GKH, a new cosmetic ingredient. J. Cosmet. Sci. 54:483-491 (2003).
27. M. P. M. Schutze-Redelmeier, S. Kong, M. B. Bally, J. P. Dutz. Antennapedia transduction sequence promotes anti tumor immunity to epicutaneously administered CTL epitopes. Vaccine 22:1985-1991 (2004).
28. A. Ho, S. R. Schwarze, S. J. Mermelstein, G. Waksman, S. Dowdy. Synthetic protein transduction domains: enhanced transduction potential in vitro and in vivo. Cancer Res. 61:474-477 (2001).
29. D. J. Tessier, P. Komalavilas, B. Liu, C. K. Kent, J. S. Thresher, C. M. Dreiza, A. Panitch, L. Joshi, E. Furnish, W. Stone, R. Fowl, C. M. Brophy. Transduction of peptides analogs of the small heat shock-related protein HSP20 inhibits intimal hyperplasia. J. Vasc. Surg., 40:106-114 (2004).
30. D. J. Tessier, P. Komalavilas, E. McLemore, J. Thresher, C. M. Brophy. Sildenafil-induced vasorelaxation is associated with increases in the phosphorylation of the heat shock-related protein 20 (HSP20). J. Surg. Res. 118: 21-5 (2004).
31. M. Foldvari, M. E. Baca-Estrada, Z., He, J. Hu, S. Attah-Poku, M. King. Dermal and transdermal delivery of protein pharmaceuticals: lipid-based delivery systems for interferon-α. Biotechnol. Appl. Biochem. 30:129-137 (1999).
32. J. D. Bos, M. M. H. M. Meinardi. The 500 Dalton rule for the skin penetration of chemical compounds and drugs. Exp. Dermatol. 9:165-169 (2000).
33. K. Moser, K. Kriwet, A. Naik, Y. N. Kalia, R. H. Guy. Passive skin penetration enhancement and its quantification in vitro. Eur. J. Pharm. Bipharm. 52:103-112 (2001).
34. R. F. V. Lopez, M. V. L. B. Bentley, M. B. Delgado-Charro, R. H. Guy. Iontophoretic delivery of 5-aminolevulinic acid (ALA): effect of pH. Pharm. Res. 18:311-315 (2001).
35. R. Alvarez-Román, A. Naik, Y. N. Kalia, H. Fessi, R. H. Guy. Visualization of skin penetration using confocal laser scanning microscopy. Eur. J. Pharm. Biopharm. 58:301-316 (2004).
36. A. C. Williams, B. W. Barry. Penetration enhancers. Adv. Drug Deliv. Rev. 56:603-618 (2004).
37. T. Ogiso, M. Ywaki, T. Paku. Effect of various enhancers on transdermal penetration of indomethacin and urea and relationship between penetration parameters and enhancement factors. J. Pharm. Sci. 84:482-488 (1995).
38. M. G. Carr, J. Corish, O. I. Corrigan. Drug delivery from a liquid crystalline base across Visking and human stratum corneum. Int. J. Pharm. 157:35-42 (1997).
39. P. B. Robbins, S. F. Oliver, S. M. Sheu, P. Goodnough, P. Wender, P. A. Khavari. Peptide delivery to tissues via reversibly linked protein transduction sequences. Biotechiques 33:190-194 (2002).
40. J. Park, J. Ryu, L. H. Jin, J. H. Bahn, J. A. Kim, C. S. Yoon, D. W. Kim, K. H. Han, W. S. Eum, H. Y. Kwon, T. C. Kang, M. H. Won, J. H. Kang, S. W. Cho, S. Y. Choi. 9-polylysine protein transduction domain: enhanced penetration efficiency of superoxide dismutase into mammalian cells and skin. Mol. Cells. 13:202-208 (2002).
41. V. H. L. Lee, Enzymatic barriers to peptide and protein absorption. Crit. Rev. Ther. Drug Carrier Syst. 5:69-97 (1988).
42. N. G. Turner, L. Ferry, M. Price, C. Cullander, R. H. Guy. Iontophoresis of L-poly-lysines: the role of molecular weight? Pharm. Res. 14:1322-1331 (1997).
43. L. J. Weimann, J. Wu. Transdermal delivery of L-poly-lysine by sonomacroporation. Ultasound Med. Biol. 28:1173-1180 (2002).
44. J. Y. Fang, W. R. Lee, S. C. Shen, H. Y. Wang, C. L. Fang, C. H. Hu. Transdermal delivery of macromolecules by erbium: YAG laser. J. Control. Rel. 100:75-85 (2004).
45. P. J. White, R. D. Fogarty, I. J. Liepe, P. M. Delaney, G. A. Werther, C. J. Wraight. Live confocal microscopy of oligonucleotide uptake by keratinocytes in human skin grafts on nude mice. J. Invest. Dermatol. 112:887-892 (1999).
46. T. F. Zioncheck, S. A. Chen, L. Richardson, M. Mora-Worms, C. Lucas, D. Lewis, J. D. Green, J. Mordenti. Pharmacokinetics and tissue distribution of recombinant human transforming growth factor beta 1 after topical and intravenous administration in male rats. Pharm. Res. 11:213-220 (1994).
47. S. Frank, B. Stallmeyer, H. Kampfer, N. Kolbe, J. Pfeilschifter. Leptin enhanes wound re-epithelization and constitutes a direct function of leptin in skin repair. J. Clin. Invest., 106:501-509 (2000).
48. K. Lintner, O. Peschard. Biologically active peptides: from a laboratory bench curiosity to function skin care product. Int. J. Cosmet. Sci. 22:207-218 (2000).
49. C. D., Partidos, A. S. Beignon, F. Mawas, G. Belliard, J. P. Briand, S. Muller. Immunity under the skin: potential application for topical delivery of vaccines. Vaccine 21:776-80 (2003).
50. H. Schaefer, T. E. Redelmeier. Skin Barrier. Principles of percutaneous absorption. Kaerger, Basel, 1996.
51. P. E. Thoren, D. Persson, E. K. Esbjorner, M. Goksor, P. Lincoln, B. Norden. Membrane binding and translocation of cell penetrating peptides. Biochemistry 43:3471-89 (2004).
52. K. Ohtake, T. Maeno, H. Ueda, H. Natsume, Y. Morimoto. Poly-L-arginine predominantly increases the paracelullar permeability of hydrophilic macromolecules across rabbit nasal epithelium in vitro. Pharm. Res. 20:153-160 (2003).
53. K. Ohtake, T. Maeno, H. Ueda, M. Ogihara, A. Natsume, Y. Morimoto. Poly-L-arginine enhances paracellular permeability via serine/threonine phosphorylation of ZO-1 and tyrosine dephosphorilation of occludin in rabbit nasal epithelium. Pharm. Res. 20:1838-1845 (2003).
54. K. Morita, Y. Myachi. Tight junctions in the skin. J. Dermatol. Sci. 31:81-89 (2003).

### Example 4

### Mucosal Delivery

Mucosal delivery was examined using fluorescently labeled WL-P20 (SEQ ID NO: 10) (1 mM in K-Y Jelly, FITC-bA-WLRRIKAWLRRApSAPLPGLK, (SEQ ID NO: 44) where bA is beta-alanine and pS is phosphoserine). Peptide was applied to both the vagina and anal canal using an applicator and allowed to penetrate for 4 hours. Tissue was excised and embedded in frozen tissue embedding medium (HistoPrep) for cryosectioning. Sections were mounted in anti-fade reagent and examined using fluorescence microscopy (Zeiss Axiovert). Mucosal penetration in the vagina was achieved, however only minimal fluorescence was observed in the anal canal. These results suggest that WL-P20 (SEQ ID NO: 10) transduction is more efficient in the vaginal than rectal mucosa.

### Example 5

### Vasorelaxation

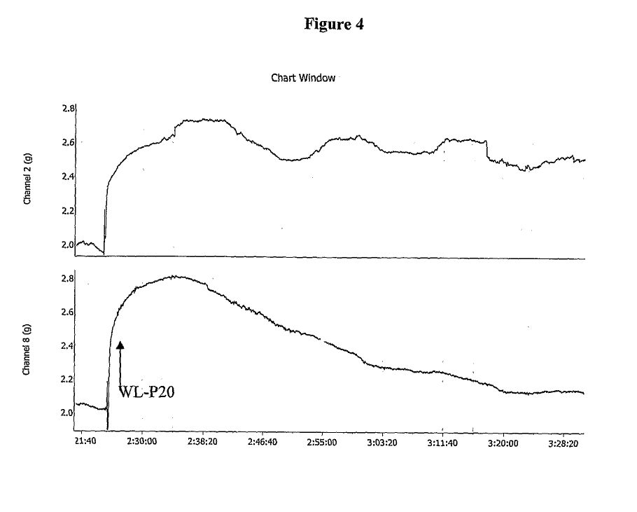

Rat aorta was isolated and dissected free from connective and fat tissue. Transverse rings, 3.0 mm in width, were cut and tied to silk suture. The tissue was suspended in a muscle bath containing a bicarbonate buffer (120 mM NaCl, 4.7 mM KCl, 1.0 mM MgSO₄, 1.0 mM NaH₂PO₄, 10 mM glucose, 1.5 mM CaCl₂, and 25 mM Na₂HCO₃, pH 7.4) and equilibrated with 95% O₂/5% CO₂ at 37° C. The rings were fixed at one end to a stainless steel wire and attached to a force transducer in muscle perfusion system (Radnotti). The rings were then progressively stretched, and the isometric force generated in response to 110 mM KCl (with equimolar replacement of NaCl in bicarbonate buffer) was determined until the consistent maximal force was produced. Agonists and peptide was added directly to the baths. Tissue that was pre-contracted with 110 mM potassium chloride (KCl) followed by 1.0 mM WL-P20 (WLRRIKAWLRRApSAPLPGLK, pS is phosphoserine) (SEQ ID NO: 10), where pS is phosphoserine) relaxed compared to untreated control (13% relaxation at 10 minutes, 50% relaxation at 30 minutes, and maximum 88% relaxation at ˜60 minutes, FIG. 4). Percent relaxation was calculated relative to maximum force generated with KCl. These data indicate that WL-P20 (SEQ ID NO: 10) relaxes tissue over a longer time course than YARAAARQARAWLRRApSAPLPGLK (SEQ ID NO: 42) (maximum relaxation achieved within 5-10 minutes). Such a difference may result from different mechanisms of penetration and/or intracellular localization.

### Example 6

### Anti-Fibrotic Activity

As a key marker of the anti-fibrotic activity of HSP20 peptides, we have previously examined expression levels of connective tissue growth factor (CTGF) in human keloid fibroblasts after stimulation with transforming growth factor beta 1 (TGFβ1). Cells were grown in 10 cm² dishes to 70% confluence in DMEM with 10% fetal bovine serum (FBS) and additional penicillin and streptomycin (1%), at 37° C. and 10% CO₂. Cells were serum starved in DMEM containing 0.5% FBS for 48 hours before the experiment. Cells were either untreated (control) or treated with TGFβ1 (2.5 ng/mL) in the presence or absence of WL-P20 (SEQ ID NO: 10) phosphopeptide (WLRRIKAWLRRApSAPLPGLK, where pS is phosphoserine) (10 or 50 μM) for 24 hours. At the end of the experiment, cells were rinsed with PBS, and homogenized using urea-dithiothreitol-chaps (UDC) buffer. Lysates were mixed, centrifuged (6000×g) for 20 minutes, and the supernatant was used for determination of protein expression. Samples (20 μg of protein) were loaded on 15% SDS-PAGE gels, and the proteins were electrophoretically transferred to Immobilon membranes. Immunoblotting with CTGF antibodies was used in conjunction with near infrared detection antibodies to determine CTGF expression (Odyssey Li-Cor, Lincoln, Nebr.). Loading differences were corrected for by normalizing to GAPDH expression

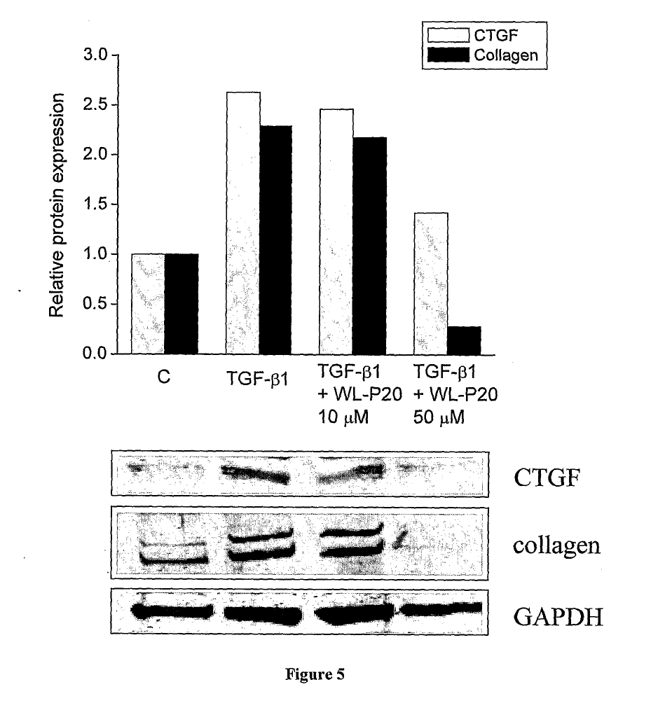

Similar to previous experiments using different transduction domains (YARAAARQARA (SEQ ID NO: 19) with WLRRApSAPLPGLK) (SEQ ID NO: 9), WL-P20 (SEQ ID NO: 10) also inhibits TGFβ1-induced CTGF and collagen expression (FIG. 5). Human keloid fibroblasts were serum-starved in DMEM medium containing 0.5% FBS for 48 hours, and treated with 2.5 ng/mL of TGF-betal for 24 hours and concomitantly treated with the WL-20 (SEQ ID NO: 10) (10 or 50 μM) for 24 hours. The Western blot bands were quantified by densitometry, and CTGF and collagen expression were related to GAPDH expression to correct for loading differences. The expression of CTGF and collagen in control cells was set to 1 for comparison of different blots.

In fact, WL-P20 (SEQ ID NO: 10) appears to more strongly inhibit the fibrotic response. For example, CTGF expression was reduced 46% with 50 μM WL-P20 compared to TGFβ1, whereas doses of 50 μM WLRRApSAPLPGLK (SEQ ID NO: 9) gave the maximal effect of 30% reduction relative to TGFβ1 treatment.

We claim:

1. An isolated polypeptide, comprising an amino acid sequence according to general formula I:

| | |
|---|---|
| (X₁X₂B₁B₂X₃B₃X₄)ₙ | (SEQ ID NO: 1) |

wherein X₁-X₄ are independently any hydrophobic amino acid;
wherein B₁, B₂, and B₃ are independently any basic amino acid; and
wherein n is between 1 and 10.

2. The isolated polypeptide of claim 1, wherein X₁-X₄ are independently any hydrophobic amino acid selected from the group consisting of Trp, Tyr, Leu, Ile, Phe, Val, Met, Cys, Pro, and Ala; and wherein B₁, B₂, and B₃ are independently arginine, histidine, or lysine.

3. The isolated polypeptide of claim 2, wherein both B₁ and B₂ are arginine or lysine and B₃ is either lysine or arginine but is not the same as B₁ and B₂.

4. The isolated polypeptide of claim 2 wherein B₁ and B₂ are arginine and B₃ is lysine.

5. The isolated polypeptide of claim 3, wherein X₁-X₄ are independently selected from the group consisting of Trp, Leu, Ile, and Ala.

6. The isolated polypeptide of claim 4, wherein X₁ is Trp, X₂ is Leu, X₃ is Ile, or X₄ is Ala, or any combination thereof.

7. The isolated polypeptide of claim 1, wherein n is 1, 2, or 3.

8. An isolated composition, comprising
(a) the isolated polypeptide of claim 1; and
(b) a cargo.

9. The isolated composition of claim 8, wherein the cargo is covalently bound to the isolated polypeptide.

10. The isolated composition of claim 8, wherein the cargo comprises an agent selected from the group consisting of therapeutic agents, diagnostic agents, prognostic agents, and imaging agents.

11. The isolated composition of claim 8, wherein the cargo comprises a molecule selected from the group consisting of polypeptides, polynucleotides, antibodies, and organic molecules.

12. The isolated composition of claim 8, wherein the cargo comprises a molecule selected from the group consisting of antipyretics, analgesics, steroidal anti-inflammatory drugs, coronary vasodilators, peripheral vasodilators, antibiotics, synthetic antimicrobials, antiviral agents, anticonvulsants, antitussives, expectorants, bronchodilators, diuretics, muscle relaxants, cerebral metabolism ameliorants, tranquilizers; beta-blockers; antiarrthymics; athrifuges; anticoagulants; liver disease drugs; anti-epileptics; antihistamines; antiemetics; depressors;. hyperlipidemia agents; sympathetic nervous stimulants, oral diabetes therapeutic drugs, oral carcinostatics, vitamins, opioids, and, angiotensin convertase inhibitors.

13. The isolated composition of claim 8, wherein the cargo comprises an HSP20 peptide.

14. The isolated composition of claim 13, wherein the HSP20 peptide comprises an amino acid sequence according to formula 1:

| | |
|---|---|
| X3-A(X4)APLP-X5 | (SEQ ID NO: 7) |

wherein X3 is 0, 1, 2, 3, or 4 amino acids of the sequence WLRR (SEQ ID NO: 8);
X4 is selected from the group consisting of S, T, Y, D, E, hydroxylysine, hydroxyproline, phosphoserine analogs and phosphotyrosine analogs;
X5 is 0, 1, 2, or 3 amino acids of a sequence of genus Z1-Z2-Z3,
wherein Z1 is selected from the group consisting of G and D;
Z2 is selected from the group consisting of L and K; and
Z3 is selected from the group consisting of K, S and T.

15. The isolated composition of claim 13, wherein the HSP20 peptide comprises an amino acid sequence according to SEQ ID NO: 9.

16. The isolated composition of claim 13, wherein the HSP20 peptide comprises an amino acid sequence according to formula 2:

| | |
|---|---|
| X2-X3-RRA-X4-AP | (SEQ ID NO: 13) |

Wherein X2 is absent or is W;
X3 is absent or is L; and
X4 is selected from the group consisting of S, T, Y, D, E, phosphoserine analogs and phosphotyrosine analogs.

17. The isolated composition of claim 13, wherein the isolated polypeptide comprises an amino acid sequence according to SEQ ID NO: 3.

18. The isolated composition of claim 17, wherein n is 1, 2, or 3.

19. A pharmaceutical composition comprising the isolated polypeptide of claim 1.

20. An isolated nucleic acid encoding the polypeptide of claim 1.

21. An isolated nucleic acid encoding the composition of claim 13.

22. An expression vector comprising DNA control sequences operatively linked to the isolated nucleic acid of claim 21.

23. Recombinant host cells comprising the expression vector of claim 22.

24. An improved biomedical device, wherein the biomedical device comprises the isolated composition of claim 8.

25. A method for in vivo delivery of active agents, comprising administering the isolated composition of claim 8 to a subject in need thereof.

26. A method for one or more of the following therapeutic uses:
(a) inhibiting smooth muscle cell proliferation and/or migration; (b) promoting smooth muscle relaxation; (c) increasing the contractile rate in heart muscle; (d) increasing the rate of heart muscle relaxation; (e) promoting wound healing; (f) reducing scar formation; (g) disrupting focal adhesions; (h) regulating actin polymerization; and (i) treating or inhibiting one or more of intimal hyperplasia, stenosis, restenosis, atherosclerosis, smooth muscle cell tumors, smooth muscle spasm, angina, Prinzmetal's angina (coronary vasospasm), ischemia, stroke, bradycardia, hypertension, pulmonary (lung) hypertension, asthma (bronchospasm), toxemia of pregnancy, pre-term labor, pre-eclampsia/eclampsia, Raynaud's disease or phenomenon, hemolytic-uremia, non-occlusive mesenteric ischemia, anal fissure, achalasia, impotence, migraine, ischemic muscle injury associated with smooth muscle spasm, vasculopathy, such as transplant vasculopathy, bradyarrythmia, bradycardia, congestive heart failure, stunned myocardium, pulmonary hypertension, and diastolic dysfunction;
wherein the method comprises administering to an individual in need thereof an effective amount to carry out the one or more therapeutic uses of the isolated composition of claim 14.

27. The method of claim 26 wherein the therapeutic use comprises treating or inhibiting intimal hyperplasia.

28. The method of claim 26 wherein the therapeutic use comprises promoting smooth muscle relaxation.

29. The method of claim 26 wherein the therapeutic use comprises promoting wound healing.

30. The method of claim 26 wherein the therapeutic use comprises reducing scar formation.

31. The method of claim 26 wherein the therapeutic use comprises treating or inhibiting vasospasm.

32. The method of claim 31 wherein the vasospasm is selected from the group consisting of angina, coronary vasospasm, Prinzmetal's angina, ischemia, stroke, bradycardia, and hypertension.

33. The method of claim 26 wherein the therapeutic use comprises treating or inhibiting a cardiac disorder selected from the group consisting of bradyarrhythmia, bradycardia, congestive heart failure, pulmonary hypertension, stunned myocardium, and diastolic dysfunction.

34. A method for topical or transdermal delivery of a cargo, comprising combining a transduction domain and a cargo, and contacting the skin of a subject to whom the active agent is to be delivered, wherein the active cargo is delivered through the skin of the subject.

35. The method of claim 34 wherein the cargo is not covalently bound to the transduction domain.

36. The method of claim 34, wherein the transduction domain comprises an isolated polypeptide according to general formula I:

| | |
|---|---|
| (X₁X₂B₁B₂X₃B₃X₄)ₙ | (SEQ ID NO: 1) |

wherein X₁-X₄ are independently any hydrophobic amino acid;
wherein B₁,B₂ and B₃ are independently any basic amino acid; and
wherein n is between 1 and 10.

37. The method of claim 34 wherein the transduction domain comprises a polypeptide selected from the group consisting of (R)₄₋₉ (SEQ ID NO: 40); GRKKRRQRRRPPQ (SEQ ID NO: 18); YARAAARQARA (SEQ ID NO: 19); DAATATRGRSAASRPTERPRAPARSASRPRRPVE (SEQ ID NO: 20); GWTLNSAGYLLGLINLKALAALAKKIL (SEQ ID NO: 21); PLSSIFSRIGDP (SEQ ID NO:22); AAVALLPAVLLALLAP (SEQ ID NO: 23); AAVLLPVLLAAP (SEQ ID NO: 24); VTVLALGALAGVGVG (SEQ ID NO: 25); GALFLGWLGAAGSTMGAWSQP (SEQ ID NO: 26); GWTLNSAGYLLGLINLKALAALAKKIL (SEQ ID NO: 27); KLALKLALKALKAALKLA (SEQ ID NO: 28); KETWWETWWTEWSQPKKKRKV (SEQ ID NO: 29); KAFAKLAARLYRKAGC (SEQ ID NO: 30); KAFAKLAARLYRAAGC (SEQ ID NO: 31); AAFAKLAARLYRKAGC (SEQ ID NO: 32); KAFAALAARLYRKAGC (SEQ ID NO: 33); KAFAKLAAQLYRKAGC (SEQ ID NO: 34); GGGGYGRKKRRQRRR (SEQ ID NO: 35); and YGRKKRRQRRR (SEQ ID NO: 36).
